SEQUENCE LISTING (8 sequences)

SEQ ID NO: 1 - DNA, 1197 bp, Human GPR54 receptor protein
atgcacaccgtggctacgtccggacccaacgcgtcctggggggcaccggccaacgcctccggctgcccgggctgtggcgccaacgcctcggacggcccagtcccttcgccgcgggccgtggacgcctggctcgtgccgctcttcttcgcggcgctgatgctgctgggcctggtggggaactcgctggtcatctacgtcatctgccgccacaagccgatgcggaccgtgaccaacttctacatcgccaacctggcggccacggacgtgaccttcctcctgtgctgtgtccccttcacggccctgctgtacccgctgcccggctgggtgctgggcgacttcatgtgcaagttcgtcaactacatccagcaggtctcggtgcaggccacgtgtgccactctgaccgccatgagtgtggaccgctggtacgtgacggtgttcccgttgcgcgccctgcaccgccgcacgccccgcctggcgctggctgtcagcctcagcatctgggtaggctctgcggcggtgtctgcgccggtgctcgccctgcaccgcctgtcacccgggccgcgcgcctactgcagtgaggccttccccagccgcgccctggagcgcgccttcgcactgtacaacctgctggcgctgtacctgctgccgctgctcgccacctgcgcctgctatgcggccatgctgcgccacctgggccgggtcgccgtgcgccccgcgcccgccgatagcgccctgcaggggcaggtgctggcagagcgcgcaggcgccgtgcgggccaaggtctcgcggctggtggcggccgtggtcctgctcttcgccgcctgctggggccccatccagctgttcctggtgctgcaggcrctgggccccgcgggctcctggcacccacgcagctacgccgcctacgcgcttaagacctgggctcactgcatgtcctacagcaactccgcgctgaacccgctgctctacgccttcctgggctcgcacttccgacaggccttccgccgcgtctgcccctgcgcgccgcgccgcccccgccgcccccgccggcccggaccctcggaccccgcagccccacacgcggagctgcwccgcctggggtcccacccggcccccgccagggcgcagaagccagggagcagtgggctggccgcgcgcgggctgtgcgtcctgggggaggacaacgcccctctctga

SEQ ID NO: 2 - protein, 398 aa, Human GPR54
MHTVATSGPNASWGAPANASGCPGCGANASDGPVPSPRAVDAWLVPLFFAALMLLGLVGNSLVIYVICRHKPMRTVTNFYIANLAATDVTFLLCCVPFTALLYPLPGWVLGDFMCKFVNYIQQVSVQATCATLTAMSVDRWYVTVFPLRALHRRTPRLALAVSLSIWVGSAAVSAPVLALHRLSPGPRAYCSEAFPSRALERAFALYNLLALYLLPLLATCACYAAMLRHLGRVAVRPAPADSALQGQVLAERAGAVRAKVSRLVAAVVLLFAACWGPIQLFLVLQALGPAGSWHPRSYAAYALKTWAHCMSYSNSALNPLLYAFLGSHFRQAFRRVCPCAPRRPRRPRRPGPSDPAAPHAELHRLGSHPAPARAQKPGSSGLAARGLCVLGEDNAPL

SEQ ID NO: 3 - DNA, 1197 bp, Human GPR54 gene; identical to SEQ ID NO: 1

SEQ ID NO: 4 - protein, 398 aa, Human GPR54 gene
  VARIANT 287, 364: Xaa = Any Amino Acid
MHTVATSGPNASWGAPANASGCPGCGANASDGPVPSPRAVDAWLVPLFFAALMLLGLVGNSLVIYVICRHKPMRTVTNFYIANLAATDVTFLLCCVPFTALLYPLPGWVLGDFMCKFVNYIQQVSVQATCATLTAMSVDRWYVTVFPLRALHRRTPRLALAVSLSIWVGSAAVSAPVLALHRLSPGPRAYCSEAFPSRALERAFALYNLLALYLLPLLATCACYAAMLRHLGRVAVRPAPADSALQGQVLAERAGAVRAKVSRLVAAVVLLFAACWGPIQLFLVLQXLGPAGSWHPRSYAAYALKTWAHCMSYSNSALNPLLYAFLGSHFRQAFRRVCPCAPRRPRRPRRPGPSDPAAPHAELXRLGSHPAPARAQKPGSSGLAARGLCVLGEDNAPL

SEQ ID NO: 5 - DNA, 1191 bp, Mouse GPR54
atggccaccgaggcgacattggctcccaatgtgacctggtgggctccgtccaacgcttcaggatgcccaggctgcggtgtcaacgcctcggatgacccaggctctgcgccaaggcccctggatgcctggctggttcccctgtttttcgctacactcatgttgcttgggctggtcggaaactcattggtcatctacgttatctgccgccacaagcacatgcagacagttaccaacttctacatcgctaacctggctgccacagacgtcactttcctactgtgctgcgtgcccttcaccgcactcctctacccgctgcccgcctgggtgctgggagacttcatgtgcaaattcgtcaactacatccagcaggtctcggtgcaagccacatgtgccactctgacggccatgagtgtggaccgctggtatgtgactgtgttcccgctgcgtgcacttcaccgccgcactccgcgcctggccctggctgtcagcctcagcatctgggtggggtcagcagctgtgtccgccccggtgctggccctgcaccgcctgtcgccagggcctcgcacctactgcagcgaggcgtttcccagccgcgccctggagcgcgccttcgcgctctacaacctgctggctctatatctgctgccgctgctcgccacctgcgcctgctacggcgccatgctgcgccacctgggccgtgcggctgtacgccccgcacccactgacggcgccctgcagggacagctgctagcacagcgcgccggagcagtgcgcaccaaggtctcccggctggtggccgctgtcgtcctgctcttcgccgcctgctggggcccgatccagctgttcctggtgcttcaagccctgggcccctcgggggcctggcaccctcgaagctatgccgcctacgcggtcaagatctgggctcactgcatgtcctacagcaactcggcgctcaatccgctgctctatgccttcctgggttcacacttcagacaggccttctgccgcgtgtgcccctgctgccggcaacgccagcgccggccccacacgtcagcgcactcggaccgagctgcaactcacactgtgccgcacagccgtgctgcgcaccctgtgcggatcaggagcccggagcctgggaaccctgtggtgcgctcgccctgcgctcagagtgaacgcactgcctcactctga

SEQ ID NO: 6 - protein, 396 aa, Mouse GPR54
MATEATLAPNVTWWAPSNASGCPGCGVNASDDPGSAPRPLDAWLVPLFFATLMLLGLVGNSLVIYVICRHKHMQTVTNFYIANLAATDVTFLLCCVPFTALLYPLPAWVLGDFMCKFVNYIQQVSVQATCATLTAMSVDRWYVTVFPLRALHRRTPRLALAVSLSIWVGSAAVSAPVLALHRLSPGPRTYCSEAFPSRALERAFALYNLLALYLLPLLATCACYGAMLRHLGRAAVRPAPTDGALQGQLLAQRAGAVRTKVSRLVAAVVLLFAACWGPIQLFLVLQALGPSGAWHPRSYAAYAVKIWAHCMSYSNSALNPLLYAFLGSHFRQAFCRVCPCCRQRQRRPHTSAHSDRAATHTVPHSRAAHPVRIRSPEPGNPVVRSPCAQSERTASL

SEQ ID NO: 7 - DNA, 1191 bp, Mouse GPR54 Gene; identical to SEQ ID NO: 5

SEQ ID NO: 8 - protein, 390 aa, Mouse GPR54 Gene
WPPRRHWLPMPGGLRPTLQDAQAAVSTPRMTQALRQGPWMPGWFPCFSLHSCCLGWSETHWSSTLSAATSTCRQLPTSTSLTWLPQTSLSYCAACPSPHSSTRCPPGCWETSCANSSTTSSRSRCKPHVPLRPVWTAGMLCSRCVHFTAALRAWPWLSASASGWGQQLCPPRCWPCTACRQGLAPTAARRFPAAPWSAPSRSTTCWLYICCRCSPPAPATAPCCATWAVRLYAPHPLTAPCRDSCHSAPEQCAPRSPGWWPLSSCSSPPAGARSSCSWCFKPWAPRGPGTLEAMPPTRSRSGLTACPTATRRSIRCSMPSWVHTSDRPSAACAPAAGNASAGPTRQRTRTELQLTLCRTAVLRTLCGSGARSLGTLWCARPALRVNALPH

(19) United States
(12) Patent Application Publication
(10) Pub. No.: US 2005/0075494 A1
(43) Pub. Date: Apr. 7, 2005

(54) Isolated nucleic acid molecules encoding a human and mouse g protein-coupled receptor-gpr54: encoded proteins, cells transformed therewith and uses thereof

(76) Inventors: Liu Qingyun, North Wales, PA (US); Michelle Clements, Sellersville, PA (US); Terence P. McDonald, Langhorne, PA (US)

Correspondence Address: MERCK AND CO., INC, P O BOX 2000, RAHWAY, NJ, 07065-0907, US

(21) Appl. No.: 10/451,002

(22) PCT Filed: Dec. 14, 2001
(86) PCT No.: PCT/US01/48333

Related U.S. Application Data
(62) Division of application No. 60/256,299, filed on Dec. 18, 2000.

(51) Int. Cl. C07H 21/04; C07K 14/705

(52) U.S. Cl. 536/23.5; 530/350; 435/69.1; 435/320.1; 435/325

(57) ABSTRACT

Disclosed herein are newly identified polynucleotides, polypeptides encoded by such polynucleotides, the use of such polynucleotides and polypeptides, as well as the production of such polynucleotides and polypeptides. More particularly, This invention relates to newly identified polynucleotides, polypeptides encoded by such polynucleotides, the use of such polynucleotides and polypeptides, as well as the production of such polypeptides by recombinant techniques. More particularly, the polynucleotides and polypeptides of the present invention relate to a G-Protein coupled receptor protein, hereinafter referred to as Human GPR54 (GPR54), which happens to be an orphan receptor protein. The invention also relates to inhibiting or activating the action of such polynucleotides and polypeptides.

### CROSS-REFERENCE TO RELATED APPLICATIONS

Not Applicable.

### STATEMENT REGARDING FEDERALLY-SPONSORED R&D

Not Applicable.

### REFERENCE TO MICROFICHE APPENDIX

Not Applicable.

### FIELD OF THE INVENTION

This invention relates to newly identified polynucleotides, polypeptides encoded by such polynucleotides, the use of such polynucleotides and polypeptides, as well as the production of such polypeptides by recombinant techniques. More particularly, the polynucleotides and polypeptides of the present invention relate to a G-Protein coupled receptor protein, hereinafter referred to as Human GPR54 (“GPR54”). The invention also relates to inhibiting or activating the action of such polynucleotides and polypeptides. Also disclosed are methods for utilizing Human GPR54 receptor proteins and polynucleotides in the design of protocols for the treatment of diseases attending a defective GPR54 receptor protein.

### BACKGROUND OF THE INVENTION

In higher eukaryotic cells, the interaction between ligands (e.g., peptide hormones, growth factors and their analogs) and their receptors is of central importance in the transmission of and response to a variety of extracellular signals. These signals take the form of growth factors, hormones, cytokines, and peptides which bind to and activate specific receptor molecules located on their external membrane. The function of these receptors is to “sense” the cell environment and supply the cell with an input signal about any changes in the environment. The activated receptors, in turn, trigger intracellular signal transduction pathways which culminate in a wide range of cellular responses affecting gene expression, protein secretion, cell cycle progression, and cell differentiation. In general, upon ligand binding, the receptors are believed to undergo a conformational change, triggering intra-cellular responses, which, in turn, result in the activation or inhibition of some cellular process(es). Ligand analogs fall into two classes: those that mimic the effect(s) of the corresponding natural ligand, termed agonists; and those that block receptor-ligand binding or the effects elicited by the natural ligand, termed antagonists.

In eukaryotic organisms such a cell environment is comprised of the neighboring cells and the function of the receptor is to allow cells to communicate with each other directly (the paracrine regulatory system) or indirectly (the endocrine regulatory system) thus achieving harmonized response of a tissue, organ or a whole organism. In prokaryotic cells, the surface localized receptors provide a means for detecting extracellular environment.

Receptors are classified into families and superfamilies on the basis of conserved structural features. It is generally believed that under selective pressure for organisms to acquire new biological functions, new receptor family members arose from duplication of existing receptor genes leading to the existence of multi-gene families. Family members thus contain vestiges of the ancestral gene and these characteristic features can be exploited in the isolation and identification of additional family members.

In eukaryotic cells, receptor molecules determine the selective response of the cell. Each type of receptor can interact only with a specific set of ligand molecules. The cells derived from the different tissues invariably express specific sets of tissue receptors.

For example, nicotinic cholinergic receptor, upon binding acetylcholine molecule, directly activates sodium channel (Claudio et al., 1987, is incorporated herein by reference). G-protein coupled receptors activate enzymes of second messenger pathways, for example, adenylate cyclase or phospholipase C with subsequent activation of cAMP or phosphoinositide cascades (Divecha and Itvine, 1995, is incorporated herein by reference). Receptor tyrosine kinases activate cascade of MEK/MAPK kinases leading to cell differentiation and proliferation (Marshall, 1995 and Herskowitz, 1995, are incorporated herein by reference)!. Cytokine receptors activate JAK/STAT cascade which in turn can regulate other pathways as well as activate gene transcription (Hill & Treisman, 1995, is incorporated herein by reference).

It is well established that many medically significant biological processes are mediated by proteins participating in signal transduction pathways that involve G-proteins and/or second messengers, e.g., cAMP (Lefkowitz, Nature 351:353-354 (1991)). Some examples of these proteins include the G-protein coupled receptor (GPCR), such as those for adrenergic agents and dopamine (Kobilka, B. K., et al., PNAS 84:46-50 (1987); Kobilka, B. K., et al., Science 238:650-656 (1987); Bunzow, J. R., et al., Nature 336:783-787 (1988)), G-proteins themselves, effector proteins, e.g., phospholipase C, adenyl cyclase, and phosphodiesterase, and actuator proteins, e.g., protein kinase A and protein kinase C (Simon, M. I., et al., Science 252:802-8 (1991)).

G-protein-coupled receptors (GPCRs) represent the single largest family of cell surface receptors involved in signal transduction (Strader et al. 1994). In humans, it is estimated that at least 1000 distinct members direct responses to a wide variety of chemical transmitters that are key controllers of such diverse physiological processes as neurotransmission, cellular metabolism, secretion, cellular differentiation and growth, as well as inflammatory and immune responses. Therefore, they represent major targets for the development of new drug candidates, with potential application in all clinical fields.

GPCRs represent the primary mechanism by which cells sense alterations in their external environment and convey that information to the cells' interior. As their name suggests, GPCRs act primarily through their activation of ubiquitous guanine nucleotide-binding regulatory proteins: the so called ‘G-proteins’. The G protein transmembrane signaling pathways consist of three proteins: receptors, G proteins and effectors. Possible relationships among seven transmembrane receptors are reviewed in Probst et al., DNA and Cell Biology 11(1): 1-20 (1992).

G-protein coupled receptors are known to share certain structural similarities and homologies (see, e-g., Gilman, A. G., Ann. Rev. Biochem. 56: 615-649 (1987), Strader, C. D. et al. The FASEB Journal 3: 1825-1832 (1989), Kobilka, B. K., et al. Nature 329:75-79 (1985) and Young et al. Cell 45: 711-719 (1986)).

The members of the GPCR superfamily are related both structurally and functionally. The G-protein coupled receptors exhibit detectable amino acid sequence similarity and all appear to share a number of structural characteristics. The signature motif of these receptors is an extracellular amino terminus; seven predominantly hydrophobic α-helical domains (of about 20-30 amino acids) connecting at least six divergent hydrophilic loops which are believed to span the cell membrane and are referred to as transmembrane domains 1-7; approximately twenty well-conserved amino acids; and a cytoplasmic carboxy terminus. The amino acid similarity among different G-protein receptors ranges from about 20% to more than 80% and receptors which recognize similar or identical ligands generally exhibit high levels of homology. The third cytosolic loop between transmembrane domains five and six is the intracellular domain responsible for the interaction with G-proteins. G-protein coupled receptors are found in numerous sites within a mammalian host.

Functionally, G-protein coupled receptors share in common the property that upon agonist binding they transmit signals across the plasma membrane through an interaction with heterotrimeric G proteins. These receptors can be grouped based on their homology levels and/or the ligands they recognize. The G-protein coupled receptors includes dopamine receptors which bind to neuroleptic drugs used for treating psychotic and neurological disorders. Other members of this family include calcitonin, adrenergic, endothelin, cAMP, adenosine, muscarinic, acetyicholine, serotonin, histamine, thrombin, kinin, follicle stimulating hormone, opsins, endothelial differentiation gene-1 receptor and rhodopsins, odorant, cytomegalovirus receptors, etc.

The superfamily of G protein-linked receptors controls many physiological functions. These receptors mediate transmembrane signaling from external stimuli (vision, taste and smell), endocrine function (pituitary and adrenal), exocrine function (pancreas), heart rate, lipolysis, and carbohydrate metabolism. Indeed, these receptors respond to a vast range of agents such as protein hormones, chemokines, peptides, small biogenic amines, lipid-derived messengers, divalent cations (e.g. a Ca2+ and even proteases such as thrombin, which activates its receptor by cleaving off a portion of the amino terminus. Finally, these receptors play an important role in sensory perception including vision and smell.

Correlated with the broad range of agents that activate these receptors is their existence in a wide variety of cells and tissue types, indicating that they play roles in a diverse range of physiological processes. It is likely, therefore, that the GPCR superfamily is involved in a variety of pathologies.

The actions of many extracellular signals are mediated by the interaction of G-protein coupled receptors (GPCRs) and guanine nucleotide-binding regulatory proteins (G proteins). G protein-mediated signaling systems have been identified in many divergent organisms, such as mammals and yeast. GPCRs respond to, among other extracellular signals, neurotransmitters, hormones, odorants and light. GPCRs are similar and possess a number of highly conserved amino acids; the GPCRs are thought to represent a large ‘superfamily’ of proteins. Individual GPCR types activate a particular signal transduction pathway; at least ten different signal transduction pathways are known to be activated via GPCRs. For example, the beta 2-adrenergic receptor (.beta.AR) is a prototype mammalian GPCR. In response to agonist binding, .beta.AR receptors activate a G protein (G.sub.s) which in turn stimulates adenylate cyclase and cyclic adenosine monophosphate production in the cell.

The binding of an agonist to the receptor promotes conformational changes in the cytoplasmic domains that lead to the interaction of the receptor with its cognate G protein(s). Agonist-promoted coupling between receptors and G proteins leads to the activation of intracellular effectors that substantially amplify the production of second messengers feeding into the signaling cascade. Since effectors are often enzymes [e.g. adenylate cyclase, which converts ATP to cAMP, or phospholipase C, which hydrolyses inositol lipids in membranes to release inositol trisphosphate, which in turn mobilizes Ca²⁺ within a cell] or ion channels, many second messenger molecules can be produced as the result of a single agonist binding event with its receptor. Changes in the intracellular levels of ions or cAMP, or both, result in the modulation of distinct phosphorylation cascades, extending through the cytosol to the nucleus, that eventually culminate in the physiological response of the cell to the extracellular stimulus. Although the overall paradigm is apparently the same for all GPCRs, the diversity of receptors, G proteins and effectors suggest a myriad of potential signaling processes.

The function of GPCR activation is to stimulate GTP/GDP exchange at G proteins. In a cell, the guanine nucleotide exchange cycle is initiated by binding of an agonist—occupied (or activated) GPCR to a heterotrimeric G-protein in the cell membrane. This stimulates the dissociation of the GDP from the α-subunit of the G-protein, thereby allowing endogenous GTP to bind in its place. This, in turn, causes dissociation of the receptor and the Gα-GTP and Gβr-subunits of the G-protein. The Gα-GTP and Gβr-subunits can each activate effectors, such as adenyl cyclase, phospholipase C, and ion channels. The Gα-GTP is inactivated by intrinsic GTPase, which hydrolyzes the GTP to GDP; Gα-GDP in turn inactivates the GβBr by binding to it, thereby resulting in an inactive GDP-containing heterotrimeric G-protein ready for the next activation cycle.

Thus, the function of each G-protein coupled receptor is to discriminate its specific ligand from the complex extracellular milieu and then to activate G-proteins to produce a specific intracellular signal. In summary, cell surface proteins, by intracellularly transmitting information regarding the extracellular environment via specific intracellular pathways induce an appropriate response to a particular stimulus. Indeed, by virtue of an array of varied membrane surface proteins, eukaryotic cells are exquisitely sensitive to their environment.

To date, more than 800 GPCRs have actually been cloned from a variety of eukaryotic species, from fungi to humans. Ssee L. F. Kolakowski in GCRDb-WWW The G Protein-Coupled Receptor DataBase World-Wide-Web Site (http://receptor.mgh.harvard.edu/GCRDBHOME html.org)]. For humans, the most represented species, about 140 GPCRs have been cloned for which the cognate ligands are also known. This number excludes the sensory olfactory receptors, of which hundreds to thousands are predicted to exist.

Many available therapeutic drugs in use today target GPCRs, as they mediate vital physiological responses, including vasodilatation, heart rate, bronchodilation, endocrine secretion, and gut peristalsis. See, e.g., Lefkcowitz et al., Ann. Rev. Biochem. 52:159 (1983). Additionally, spontaneous activation of GPCRs occurs, where a GPCR cellular response is generated in the absence of a ligand. Increased spontaneous activity can be decreased by antagonists of the GPCR (a process known as inverse agonism); such methods are therapeutically important where diseases cause an increase in spontaneous GPCR activity.

While the structural motifs that characterize a GPCR can be recognized in the predicted amino acid sequence of a novel receptor, the endogenous ligand that activates the GPCR cannot necessarily be predicted from its primary structure. For example, over the past decade, cloning experiments have succeeded in identifying many GPCRs for which endogenous ligands are known. The search for novel GPCR genes has also identified a large cohort of genes whose products are members of the GPCR family but for which the ligands are not known. The existence of these G protein-coupled receptors demonstrates that many neurotransmitter-receptor systems remain to be identified and functionally characterized.

Recently, a new G protein-coupled receptor (GPR 54 receptor) was identified, which is a member of a subgroup of the G protein-coupled receptor superfamily.

Significantly, the newly discovered GPR54 is related to the galanin receptor family in that the polypeptide sequence of rat GPR54 is most similar to that of the galanin receptor.

The neuro-peptide galanin was isolated in 1983 from porcine upper intestine and was found to contain 29 amino acid residues (Tatemoto, K., et al, FEBS Lett., 164 124-128(1983)). The sequences of galanin from two other mammals, rat and cow, have been described (Vrontakis M. E., et al, J. Biol. Chem. 262: 16755-16758(1987); Kaplan L. M. et al, Proc. Natl. Acad. Sci. U.S.A. 85: 1065-1069 (1988) and Rokaeus, Ang. and Carlquist M., FEBS Lett. 234: 400-406 (1988)). A comparison of the peptide sequence of galanin from the mammals rat, porcine and bovine reveals that the N-terminal amino acids 1-15 are identical. Thus, it is most likely that this conserved region will be found in galanin from other mammals, including man. Galanin shows 90% homology between the species but little similarity to other known peptides.

The distribution of galanin receptors in the CNS generally complements that of galanin peptide, with high levels of galanin binding observed in the hypothalamus, amygdala, hippocampus, brainstem and dorsal spinal cord (Skofitsch et al., Peptides 7:1029-1042 (1986); Merchenthaler et al., Prog. Neurobiol. 40:711-769 (1993); see Bartfai et al., Proc. Natl. Acad. Sci. U.S.A 88:11287-11291. (1993)). Accordingly, agents modulating the activity of galanin receptors would have multiple potential therapeutic applications in the CNS. One of the most important of these is the regulation of food intake. Data from research indicates that specific receptors in the hypothalamus mediate the effects of galanin on feeding behavior, and further suggest that agents acting at hypothalamic galanin receptors may be therapeutically useful in the treatment of human eating disorders. See Kyrkouli, et al. Peptides 11:995-1001 (1990); Crawley et al., Brain. Res. 600:268-272. (1993).

Galanin receptors elsewhere in the CNS may also serve as therapeutic targets. In the spinal cord galanin is released from the terminals of sensory neurons as well as spinal interneurons and appears to play a role in the regulation of pain threshold (Wiesenfeld-Hallin et al., Proc. Natl. Acad. Sci. U.S.A 89:3334-3337 1992).

Intrathecal galanin potentiates the anti-nociceptive effects of morphine in rats and produces analgesia when administered alone (Wiesenfeld-Hallin et al., Acta Physiol. Scand. 147:457-458. 1993; Post et al., Acta Physiol. Scand. 132:583. 1988); galanin receptor agonists may therefore be useful as analgesic agents in the spinal cord.

A galanin receptor cDNA was recently isolated by expression cloning from a human Bowes melanoma cell line (Habert-Ortoli et al., Proc. Natl. Acad. Sci. U.S.A 91:9780-978 1994). The pharmacological profile exhibited by this receptor (GALR1) is similar to that observed in brain and pancreas. The cloned human receptor binds native human, porcine and rat galanin with sufficient affinity. The GALR1 receptor appears to couple to inhibition of adenylate cyclase.

Recently the rat homologue of GALR1 was cloned from the RIN14B pancreatic cell line (Burgevin, et al., J. Molec. Neurosci. 6:33-41 (1995); Parker et al., Mol. Brain Res. 34:179-189 (1996)). Importantly, the pharmacologic data reported to date suggest a substantial similarity in the pharmacological properties of the rat and human GALR1 receptors. Accordingly, it is not seen why the same would not apply to another member of the G protein-coupled receptor superfamily such as the novel GPR54 receptor proteins of the invention.

Importantly, the polypeptide sequence of rat GPR54 is most similar to that of galanin receptors. As such, it is hypothesized that pathophysiological disorders proposed to be linked to galanin receptor activation which include, inter alia, eating disorders, diabetes, pain, depression, ischemia, Alzheimer's disease and reproductive disorders may also be associated with a defective human GPR54 receptor protein. Accordingly, treatment of such disorders may be effected by the administration of GPR54 receptor-selective compounds. As such, there are many potential pharmacological uses for compounds that interact with and modulate the activity of cell surface proteins such as the GPR54 receptor proteins of the invention.

Likewise, GPR54 receptors proteins may, like the GALR2 receptors present in rat brain, may play a role in cognition, analgesia, sensory processing (olfactory, visual), processing of visceral information, motor coordination, modulation of dopaminergic activity, neuroendocrine function, sleep disorders, migraine, and anxiety.

Accordingly, applicants have endeavored to clone a human and mouse GPR54 receptor which will prove useful in target-based drug design programs. The identification of GPR54 specific therapeutic agents will be greatly facilitated by the cloning, expression, and characterization of a human and mouse GPR54 receptor.

In order to study the function of human GPR54 and to obtain disease-specific pharmacologically active agents, there is a need to obtain isolated (preferably purified) human GPR54, and isolated (preferably purified) human GPR54. In addition, there is also a need to develop assays to identify such pharmacologically active agents.

Applicants now report the isolation by expression cloning of a novel human and mouse G protein-coupled receptor—GPR54 receptor. Applicants believe that the newly discovered isolated nucleic acid molecules that encode human and mouse GPR54 will fulfill the above referenced voids in the prior art and will provide detailed information of the human and mouse GPR54 structure and function based on predictions drawn from other receptors from the G protein-coupled receptor superfamily. This, in turn, will allow for the development of therapeutic candidates effective to treat various disorders attending a defective human GPR54 or its respective receptor, etc.

The disclosed sequences and encoded proteins will provide the means for screening for GPR54 mediated disorders. As well, the identity of a human GPR54 will enable the rapid screening of a large number of compounds to identify those candidates suitable for further, in-depth studies of therapeutic applications. The compound identified above may serve for eating disorders, diabetes, pain, depression, ischemia, and Alzheimer's disease.

Hitherto no human and mouse GPR54 antagonists, have been reported. The instant application provides the means for identifying both human and mouse GPR54 antagonists that would find use in determining the physiological significance of GPR54 and to develop pharmaceutical preparations for the regulation of the physiological function of this receptor.

### SUMMARY OF THE INVENTION

This invention provides a recombinant nucleic acid molecules comprising a sequence of nucleotides that a mammalian GPR54 receptor protein, wherein the mammalian receptor-encoding nucleic acid comprises (a) the sequences of nucleotides as set forth in one of SEQ ID NOs: 1 or 4, (b) a sequence of nucleotides that hybridizes under high stringency conditions to (c) a nucleic acid encoding a human GPR54 and having a sequence identical to the sequence of the human oGPCR-encoding nucleic acid as set forth in SEQ ID NO: 1 or (d) a nucleic acid encoding a mouse GPR54 and having a sequence identical to the sequence of the mouse GPR54-encoding nucleic acid as set forth in SEQ ID NO: 4.

This invention further provides recombinant nucleic acids comprising nucleic acid molecules encoding a human GPR54 receptor protein, wherein the human receptor comprises an amino acid sequence identical to the sequence of the human receptor encoded by the shortest open reading frame indicated in SEQ ID NO: 2.

This invention further provides recombinant nucleic acids comprising nucleic acid molecules encoding a mouse GPR54 receptor protein, wherein the human receptor comprises an amino acid sequence identical to the sequence of the human receptor encoded by the shortest open reading frame indicated in SEQ ID NO: 5.

One aspect of the invention is directed to a human GPR54 agonist.

Plasmids containing genomic DNA, cDNA or mRNA encoding the invention human receptor are also provided. As are plasmids containing genomic DNA, cDNA or mRNA encoding mouse GPR54 receptor protein.

Recombinant cells containing the above-described DNAs, mRNA or plasmids i.e., encoding human GPR54 receptor protein are also provided herein.

Recombinant cells containing the above-described DNAs, mRNA or plasmids i.e., encoding mouse GPR54 receptor protein are also provided herein.

The invention also provides antisense analogs thereof and biologically active and diagnostically or therapeutically useful fragments thereof.

In accordance with a further aspect of the present invention, there are provided processes for producing the invention receptor protein(s) by recombinant techniques comprising culturing transformed prokaryotic and/or eukaryotic host cells, containing nucleic acid sequences encoding the invention receptor protein under conditions promoting expression of the invention receptor protein, followed by subsequent recovery of the polypeptide(s).

In accordance with yet another aspect of the present invention, there are provided antibodies against the invention receptor protein.

In accordance with still another embodiment of the invention, there are provided processes of administering compounds comprising the human receptor protein to a host that activates the G protein-coupled receptor signally pathway attending the disclosed human receptor.

Methods of identifying ligands that bind the receptor proteins are also provided.

In accordance with yet another aspect of the present invention, there are provided nucleic acid probes comprising nucleic acid molecules of sufficient length to specifically hybridize to the polynucleotide sequences disclosed herein. The nucleic acid probes of the invention enable one of ordinary skill in the art of genetic engineering to identify and clone similar polypeptides from any species thereby expanding the usefulness of the sequences of the invention. As well, the sequences of the invention will enable one skilled in the art to screen for and identify ligands of the disclosed GPR54 receptor protein(s) in humans and other mammalian species.

Methods for screening and the quantitative characterization of potentially pharmacologically effective compounds that specifically interact with and modulate the activity of cell membrane receptors, ion pumps and ion channels using living cells are also an object of the present invention.

Yet another aspect of the invention relates to diagnostic assays for detecting diseases associated with inappropriate Human GPR54 activity or levels.

It is yet another object of this invention to provide methods to characterize cell receptor pattern for particular cell source tissue.

It is yet another object of the invention to determine the pattern of cell surface receptors expressed in one or more cell types.

It is yet another object of the invention to confirm that a test compound influences the activity of a particular receptor.

A particularly useful application of the invention sequences is the ability to prepare synthetic receptors which are substantially free of contamination from other, potentially competing proteins. Thus, a cell transformed with the invention polynucleotide sequences could express a synthetic receptor consisting essentially of Human GPR54, which may be useful for a variety of applications, e.g., as part of an assay system free of the interferences frequently present in prior art assay systems employing non-human receptors or human tissue preparations.

Furthermore, testing of the invention receptor protein(s) with a variety of potential agonists or antagonists would provide additional information with respect to the function and activity of the respective receptor proteins. Such information may lead to the identification of compounds which are capable of very specific interaction with one or more of the receptor proteins disclosed herein. Such specificity may prove of great value in medical application.

In another aspect, the invention provides means for regulating the receptor protein-ligand interaction, and thus treating, therapeutically and/or prophylactically, a disorder which can be linked directly or indirectly to the receptors disclosed herein. By virtue of having the receptor of the invention, agonists or antagonists may be identified which stimulate or inhibit the interaction of the receptor(s) of the invention with a ligand. With either agonists or antagonists the metabolism and reactivity of cells which express the receptor proteins are controlled, thereby providing a means to abate or in some instances prevent the disease of interest.

In accordance with the above, there are provided methods of screening for compounds which bind to and activate (agonist) or inhibit activation (antagonist) of mouse or Human GPR54 receptor proteins, and for their ligands.

Thus, the invention provides screening procedures for identifying agonists or antagonists of events mediated by ligand-GPR54 receptor interaction. Such screening assays may employ a wide variety of formats, depending to some extent on which aspect of the ligand/receptor/G protein interaction is targeted.

For example, such assays may be designed to identify compounds which bind to the receptor and thereby block or inhibit interaction of the receptor with a ligand. Other assays can be designed to identify compounds which can substitute for ligand and therefore stimulate GPR54-mediated intracellular pathways.

Yet other assays can be used to identify compounds which inhibit or facilitate the association of GPR54 receptor to G protein and thereby mediate the cellular response to GPR54 receptor ligand.

In particular, the preferred method for identifying agonist or antagonist of a Human or mouse GPR54 receptor protein comprises:
contacting a cell expressing on the surface thereof the receptor protein, wherein the receptor is associated with a second component capable of providing a detectable signal in response to the binding of a compound to the receptor, with a compound to be screened under conditions favoring binding of the compound top the receptor protein; and
determining whether the compound binds to and activates or inhibits the receptor protein by measuring the level of a signal generated from the interaction of the compound with the receptor protein.

In another embodiment of the method for identifying agonist or antagonist of a human or mouse GPR54 receptor protein comprises:
determining the inhibition of binding of a ligand to cells which express the receptor proteins of the invention on the surface thereof, or to cell membranes containing the receptor proteins, in the presence of a candidate compound under conditions to permit binding to the receptor, and determining the amount of ligand bound to the receptor, such that a compound capable of causing reduction of binding of a ligand is an agonist or antagonist.

Further the present invention relates to treating conditions associated with Human GPR54 imbalance with the identified compounds.

In accordance with one aspect of the present invention, assay methods have been developed for the ready determination of the presence of functional Human or mouse GPR54 receptor. Thus, cells transformed with invention DNA or RNA sequences, or cell-lines derived from a variety of other sources can be readily screened to determine if functional receptors are produced thereby.

In another aspect, the invention features assays for detecting the invention receptor protein.

It is yet an additional object of the invention to determine the activity of a given receptor in a variety of cell types in which it is expressed.

In accordance with still another aspect of the present invention, there are provided diagnostic assays for detecting diseases related to mutations in the nucleic acid sequences encoding the invention receptor proteins and for detecting an altered level of the encoded polypeptide.

In accordance with yet a further aspect of the present invention, there are provided processes for utilizing the invention receptor protein or nucleic acid molecules encoding such polypeptides for in vitro purposes such as synthesis of DNA and manufacture of DNA vectors.

A further aspect of the invention provides assay(s) for screening and identifying potential pharmaceutically effective compounds that specifically interact with and modulate the activity of cell surface proteins, particularly the disclosed GPR54 receptor proteins.

Also within the invention is a therapeutic composition including, in a pharmaceutically-acceptable carrier, (a) the invention receptor protein, (b) an immunologically active or biologically active fragment thereof, or (c) an antibody having affinity for (a) or (b) above. These therapeutic compositions provide a means for treating various disorders characterized by abnormal (low or ubiquitous) level of the invention receptor protein or a dysfunctional receptor protein.

The DNA, mRNA, vectors, and cells provided herein permit production of human GPR54 receptor, as well as antibodies to the receptor protein. This provides a means to prepare synthetic or recombinant receptor proteins that are substantially free of contamination from many other proteins whose presence can interfere with analysis of a single human GPR54 receptor. The availability of desired receptors, i.e., GPR54 makes it possible to observe the effect of a drug substance on the receptor and to thereby perform initial in vitro screening of the drug substance in a test system that is specific for the invention receptor protein and its corresponding receptor.

These invention nucleic acids, invention receptor proteins and antibodies, including fragments thereof are useful as diagnostics, for distinguishing disease states caused by a dysfunctional endogenous Human GPR54 receptor from those which are not.

The availability of Human GPR54-specific antibodies also makes possible the application of the technique of immunohistochemistry to monitor the distribution and expression density of the invention receptor protein as well as its corresponding ligand (e.g., in normal vs diseased brain tissue). Such antibodies could also be employed for diagnostic and therapeutic applications. This antibody is preferably capable of neutralizing a biological activity of the receptor protein (i.e. adenylate cyclase activation).

Thus, antibodies, (monoclonal or polyclonal), including purified preparations of an antibody, which is capable of forming an immune complex with the invention receptor protein, such antibody being generated by using as antigen either a receptor protein or a fragment thereof.

The ability to screen drug substances in vitro to determine the effect of the drug on native human GPR54 receptor or its binding to its native ligand (agonist or antagonist) should permit the development and screening of GPR54-specific or disease-specific drugs.

Also, testing of the invention receptor protein with a variety of potential agonists or antagonists provides additional information with respect to the function and activity of the invention receptor protein and should lead to the identification and design of compounds that are capable of very specific interaction with native Human GPR54 or its interaction with a ligand. The resulting drugs should exhibit fewer unwanted side effects than drugs identified by screening with cells that express a non-Human GPR54.

Further in relation to drug development and therapeutic treatment of various disease states, the availability of polynucleotides encoding the invention receptor protein(s), in particular the human receptor, enables identification of any alterations in such genes (e.g., mutations) which may correlate with the occurrence of certain disease states. In addition, the creation of animal models of such disease states becomes possible, by specifically introducing such mutations into synthetic DNA sequences which can then be introduced into laboratory animals or in vitro assay systems to determine the effects thereof.

At least some of these and other objects are addressed by the various embodiments of the invention disclosed herein. Other features and advantages of the invention will be apparent to those of skill in the art upon further study of the specification and claims.

### DETAILED DESCRIPTION OF THE FIGURES

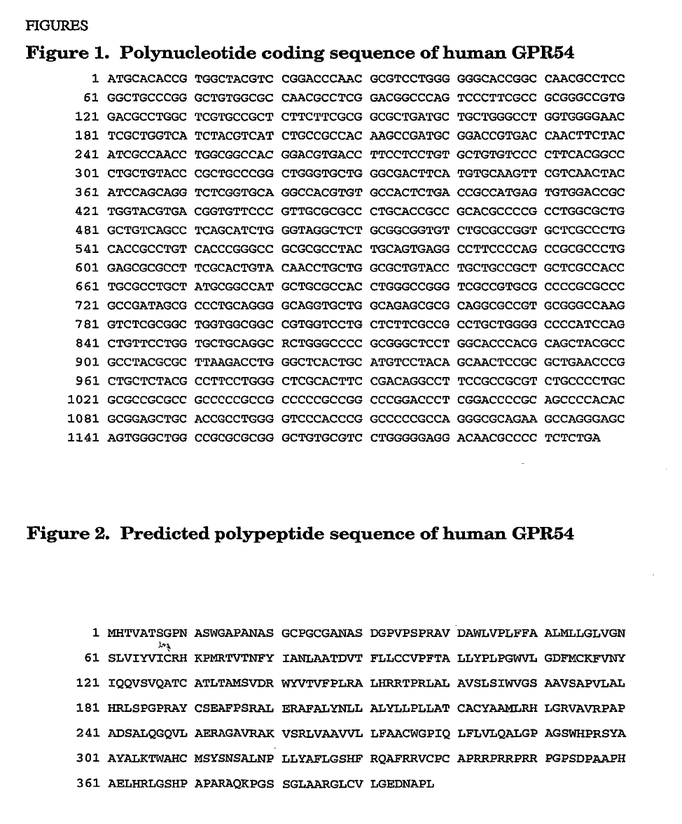

FIG. 1 presents the nucleotide sequence encoding a human GPR54.

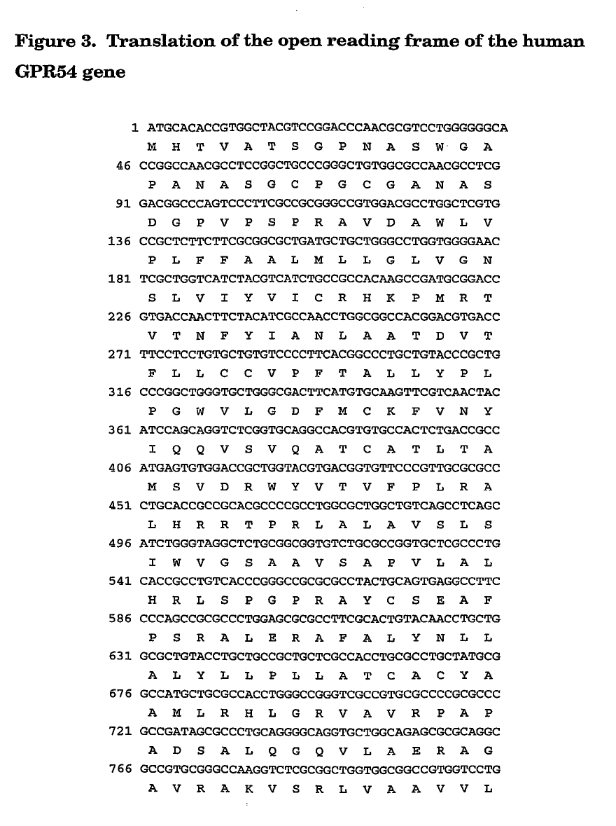

FIG. 2 presents the deduced amino acid sequence of human GPR54.

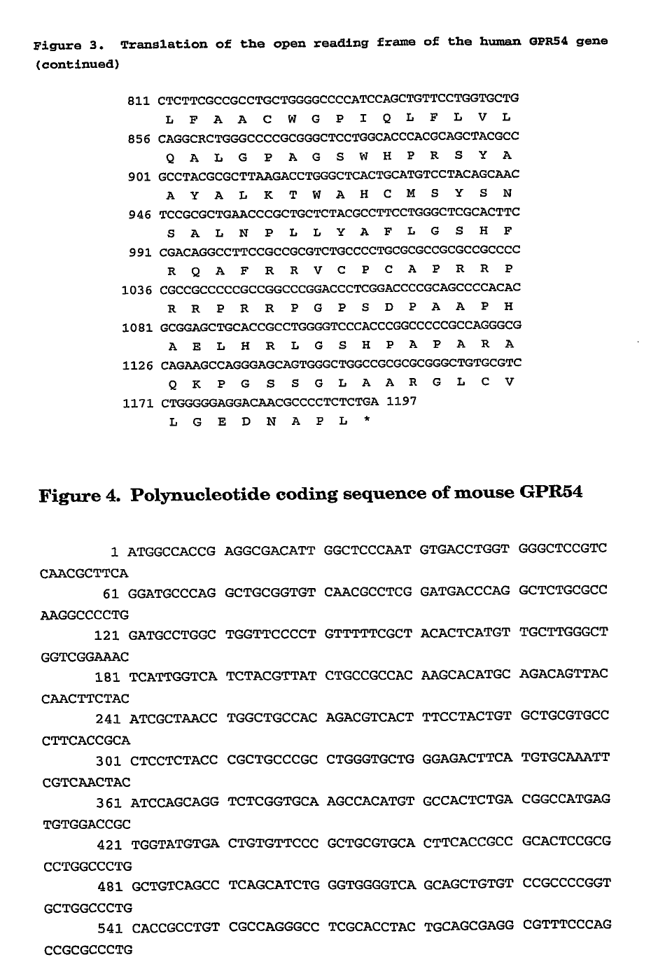

FIG. 3 presents the translation sequence of the open reading frame of the gene encoding human GPR54.

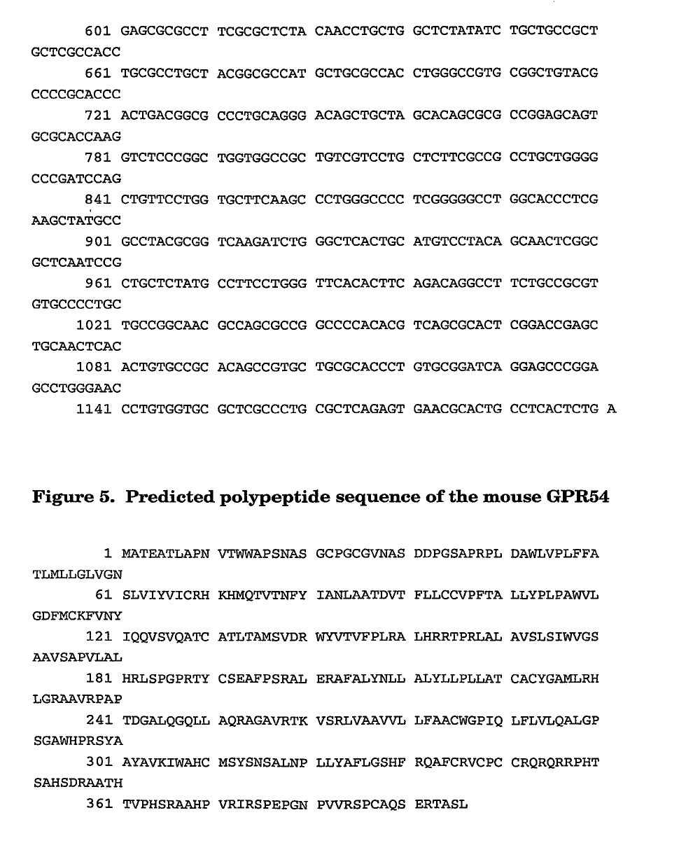

FIG. 4 presents the nucleotide sequence of the gene encoding mouse GPR54.

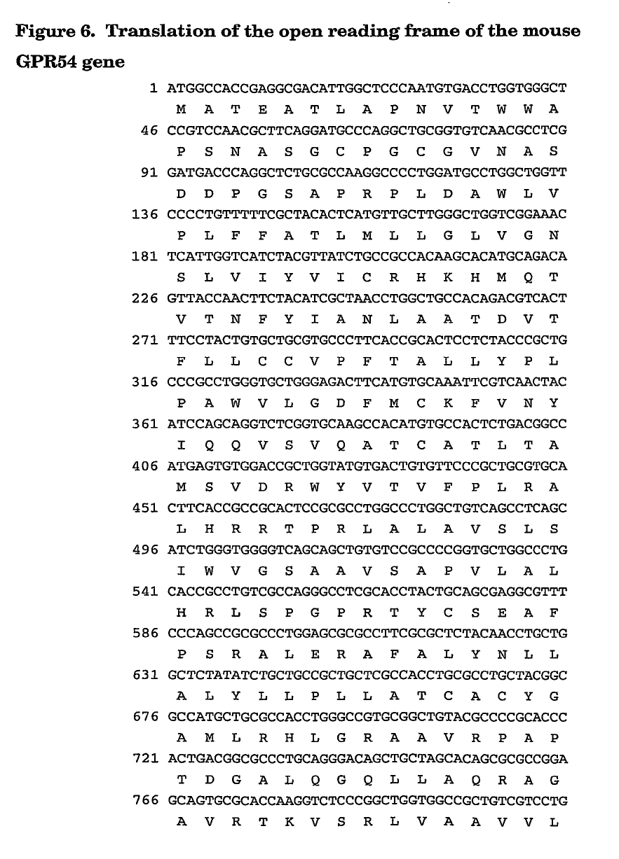

FIG. 5 presents the deduced amino acid sequence of the gene encoding mouse GPR54.

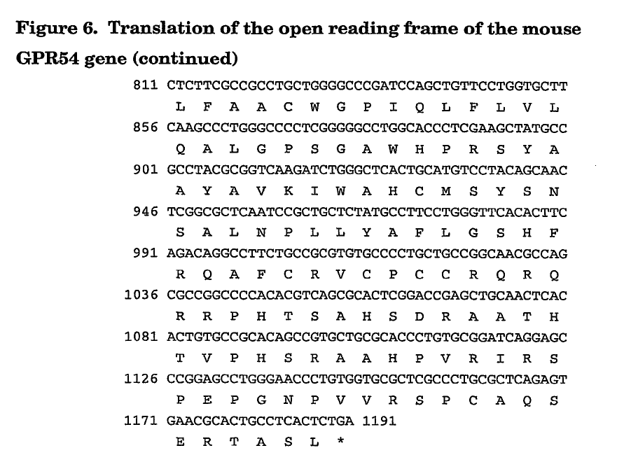

FIG. 6 presents the translation sequence of the open reading frame of the gene encoding mouse GPR54.

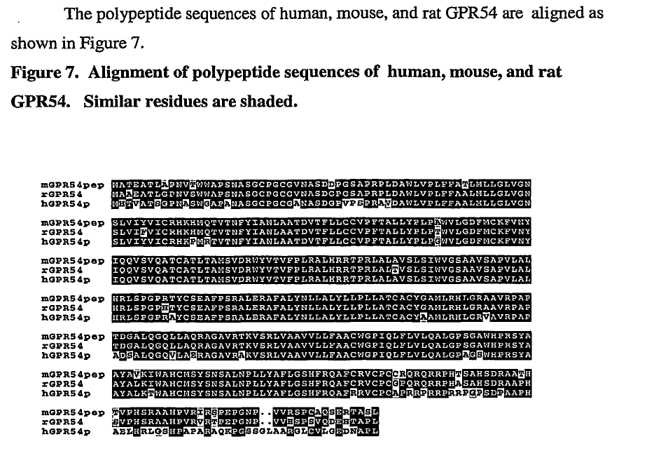

FIG. 7 depicts the polypeptide alignment of human GPR54 receptor protein and its corresponding mouse and rat equivalent.

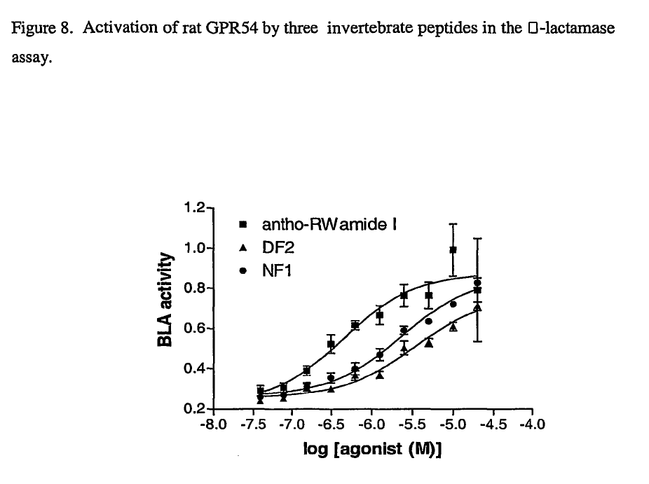

FIG. 8 depicts the dose-response curve of rat GPR54 when challenged by invertebrate peptides in a β-lactamase assay.

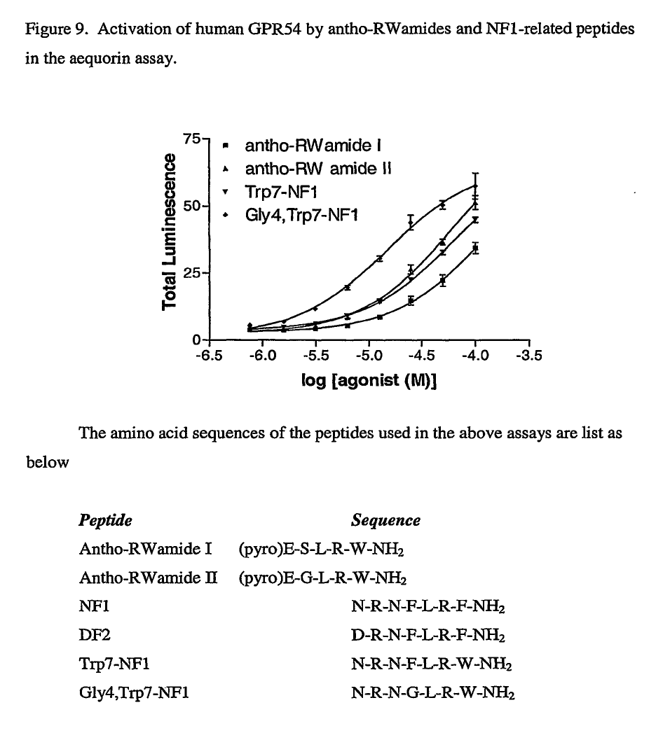

FIG. 9 depicts the dose-response curve of human GPR54 when challenged by antho-RWamides and NF1-related peptides in an aequorin assay.

### DETAILED DESCRIPTION OF THE INVENTION

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described.

All publications mentioned herein are incorporated herein by reference for the purpose of describing and disclosing the methodologies, vectors etc which are reported in the publications that might be used in connection with the invention. Nothing herein is to be construed as an admission that the invention is not entitled to antedate such disclosure by virtue of prior invention.

In the description that follows, a number of terms used in the field of recombinant DNA technology are extensively utilized. In order to provide a clearer and consistent understanding of the specification and claims, including the scope to be given such terms, the following definitions are provided.

A “gene” refers to a nucleic acid molecule whose nucleotide sequence codes for a polypeptide molecule. Genes may be uninterrupted sequences of nucleotides or they may include such intervening segments as introns, promoter regions, splicing sites and repetitive sequences. A gene can be either RNA or DNA. A preferred gene is one that encodes the invention receptor protein.

The term “nucleic acid” or “nucleic acid molecule” is intended for ribonucleic acid (RNA) or deoxyribonucleic acid (DNA), probes, oligonucleotides, fragment or portions thereof, and primers. DNA can be either complementary DNA (cDNA) or genomic DNA, e.g. a gene encoding the invention receptor protein. Nucleic acid refers to DNA, RNA or cDNA.

Unless otherwise indicated, a nucleotide defines a monomeric unit of DNA or RNA consisting of a sugar moiety (pentose), a phosphate group, and a nitrogenous heterocyclic base. The base is linked to the sugar moiety via the glycosidic carbon (1′ carbon of the pentose) and that combination of base and sugar is a nucleoside. When the nucleoside contains a phosphate group bonded to the 3′ or 5′ position of the pentose, it is referred to as a nucleotide. A sequence of operatively linked nucleotides is typically referred to herein as a “base sequence” or “nucleotide sequence”, and their grammatical equivalents, and is represented herein by a formula whose left to right orientation is in the conventional direction of 5′-terminus to 3′-terminus.

Each “nucleotide sequence” set forth herein is presented as a sequence of deoxyribonucleotides (abbreviated A, G, C and T). However, by “nucleotide sequence” of a nucleic acid molecule is intended, for a DNA molecule or polynucleotide, a sequence of deoxyribonucleotides, and for an RNA molecule or polynucleotide, the corresponding sequence of ribonucleotides (A, G, C and I), where each thymidine deoxyribonucleotide (T) in the specified deoxyribonucleotide sequence is replaced by the ribonucleotide uridine (U). For instance, reference to an RNA molecule having the sequence of SEQ ID NO: 1 set forth using deoxyribonucleotide abbreviations is intended to indicate an RNA molecule having a sequence in which each deoxyribonucleotide A, G or C of SEQ ID NO: 1 has been replaced by the corresponding ribonucleotide A, G or C, and each deoxyribonucleotide T has been replaced by a ribonucleotide U.

Use of the terms “isolated” and/or “purified” in the present specification and claims as a modifier of DNA, RNA, polypeptides or proteins means that the DNA, RNA, polypeptides or proteins so designated have been produced in such form by the hand of man, and thus are separated from their native in vivo cellular environment. As a result of this human intervention, the recombinant DNAs, RNAs, polypeptides and proteins of the invention are useful in ways described herein that the DNAs, RNAs, polypeptides or proteins as they naturally occur are not.

Similarly, as used herein, “recombinant” as a modifier of DNA, RNA, polypeptides or proteins means that the DNA, RNA, polypeptides or proteins so designated have been prepared by the efforts of human beings, e.g., by cloning, recombinant expression, and the like. Thus as used herein, recombinant proteins, for example, refers to proteins produced by a recombinant host, expressing DNAs which have been added to that host through the efforts of human beings.

As used herein, “mammalian” refers to the variety of species from which the invention receptor protein protein, e.g., human, rat, mouse, rabbit, monkey, baboon, chicken, bovine, porcine, ovine, canine, feline, and the like. A preferred GPR54 protein herein, is Human GPR54.

A “fragment” of a nucleic acid molecule or nucleotide sequence is a portion of the nucleic acid that is less than full-length and comprises at least a minimum length capable of hybridizing specifically with the nucleotide sequence of SEQ ID NO: 1 under stringent hybridization conditions. The length of such a fragment is preferably 15-17 nucleotides or more.

A “variant” nucleic acid molecule or DNA molecule refers to DNA molecules containing minor changes in the native nucleotide sequence encoding the invention polypeptide(s), i.e., changes in which one or more nucleotides of a native sequence is deleted, added, and/or substituted, preferably while substantially maintaining the biological activity of the native nucleic acid molecule. Variant DNA molecules can be produced, for example, by standard DNA mutagenesis techniques or by chemically synthesizing the variant DNA molecule or a portion thereof. Generally, differences are limited so that the nucleotide sequences of the reference and the variant are closely similar overall and, in many regions, identical.

Changes in the nucleotide sequence of a variant polynucleotide may be silent. That is, they may not alter the amino acids encoded by the polynucleotide. Where alterations are limited to silent changes of this type, a variant will encode a polypeptide with the same amino acid sequence as the reference.

Alternatively, the changes may be “conservative.” Conservative variants are changes in the nucleotide sequence that may alter the amino acid sequence of a polypeptide encoded by the reference polynucleotide. Such nucleotide changes may result in amino acid substitutions, additions, deletions, fusions and truncations in the polypeptide encoded by the reference sequence. Thus, conservative variants are those changes in the protein-coding region of the gene that result in conservative change in one or more amino acid residues of the polypeptide encoded by the nucleic acid sequence, i.e. amino acid substitution.

An “insertion” or “addition”, as used herein, refers to a change in an amino acid or nucleotide sequence resulting in the addition of one or more amino acid or nucleotide residues, respectively, as compared to the naturally occurring molecule.

A “substitution”, as used herein, refers to the replacement of one or more amino acids or nucleotides by different amino acids or nucleotides, respectively.

Preferably, a variant form of the preferred nucleic acid molecule has at least 70%, more preferably at least 80%, and most preferably at least 90% nucleotide sequence similarity with the native gene encoding the invention receptor protein.

“Primer” or “nucleic acid polymerase primer(s)” refers to an oligonucleotide, whether natural or synthetic, capable of acting as a point of initiation of DNA synthesis under conditions in which synthesis of a primer extension product complementary to a nucleic acid strand is initiated, i.e., in the presence of four different nucleotide triphosphates and an agent for polymerization (i.e., DNA polymerase or reverse transcriptase) in an appropriate buffer and at a suitable temperature. The exact length of a primer will depend on many factors, but typically ranges from 15 to 25 nucleotides. Short primer molecules generally require cooler temperatures to form sufficiently stable hybrid complexes with the template. A primer need not reflect the exact sequence of the template, but must be sufficiently complementary to hybridize with a template. A primer can be labeled, if desired.

Nucleic acid amplification techniques, which are well known in the art, can be used to locate splice variants of the invention receptor protein. This is accomplished by employing oligonucleotides based on DNA sequences surrounding divergent sequence(s) as primers for amplifying human RNA or genomic DNA. Size and sequence determinations of the amplification products can reveal the existence of splice variants. Furthermore, isolation of human genomic DNA sequences by hybridization can yield DNA containing multiple exons, separated by introns that correspond to different splice variants of transcripts encoding the invention receptor protein. Techniques for nucleic-acid manipulation are described generally in, for example, Sambrook et al. (1989) and Ausubel et al. (1987, with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers.

“Human GPR54” or “hGPR54 receptor protein” refers generally to a G protein-coupled receptor protein having the amino acid sequence set forth in SEQ ID NO:2, or an allelic variant thereof.

“Mouse GPR54” or “mouse GPR54 receptor protein” refers generally to a G protein-coupled receptor protein having the amino acid sequence set forth in SEQ ID NO:4, or an allelic variant thereof.

“Human GPR54 polynucleotides” refers to polynucleotides (DNA or RNA) containing a nucleotide sequence which encodes a human GPR54 receptor protein or fragment thereof, or a sequence of nucleotides that hybridize under high stringency conditions to the nucleotide sequences disclosed herein. Such nucleic acid molecule can be characterized in a number of ways, for example—the nucleic acid molecule may encode the amino acid sequence set forth in SEQ ID NO:2, or a nucleotide sequence which has at least 75.9% identity to a nucleotide sequence encoding the polypeptide of SEQ ID NO:2 or the corresponding fragment thereof, or a nucleotide sequence which has sufficient identity to a nucleotide sequence contained in SEQ ID NO: 1 or allelic variants thereof, splice variants thereof and/or their complements.

“Invention receptor protein(s)” refers to either the Human GPR54 or the Mouse GPR54. While the majority of the details appearing herein refer to Human GPR54 receptor protein, it is to be understood that that the Mouse GPR54 defined by the referenced nucleic acid molecules are also the subject matter of the invention and the details attending the Human GPR54 apply to the Mouse GPR54 as well.

“Invention nucleic acid(s)” and “nucleic acid molecules” are used interchangeably and refer to the nucleic acid molecules set forth herein.

“Receptor Activity” or “Biological Activity of the Receptor” refers to the metabolic or physiologic function of the hoGPCR including similar activities or improved activities or these activities with decreased undesirable side-effects. Also included are antigenic and immunogenic activities of the hGPR54.

“Reporter” molecules are those radionuclides, enzymes, fluorescent, chemiluminescent, or chromogenic agents which associate with, establish the presence of, and may allow quantification of a particular nucleotide or amino acid sequence.

“Antibodies” as used herein includes polyclonal and monoclonal antibodies, chimeric, single chain, and humanized antibodies, as well as Fab fragments, including the products of an Fab or other immunoglobulin expression library.

As used herein, a “splice variant” refers to variant invention receptor protein(s)-encoding nucleic acid(s) produced by differential processing of primary transcript(s) of genomic DNA, resulting in the production of more than one type of mRNA. cDNA derived from differentially processed primary transcript will encode the invention receptor protein(s) that have regions of complete amino acid identity and regions having different amino acid sequences. Thus, the same genomic sequence can lead to the production of multiple, related mRNAs and proteins. Both the resulting mRNAs and proteins are referred to herein as “splice variants”.

As used herein, a nucleic acid “probe” is single-stranded DNA or RNA, or analog thereof, that has a sequence of nucleotides that includes at least 14, preferably at least 20, more preferably at least 50, contiguous bases that are the same as or the complement of any 14 or more contiguous bases set forth in any of SEQ ID Nos:1 or 4. In addition, the entire cDNA encoding region of the entire sequence corresponding to SEQ ID NoO:1 or 4 may be used as a probe.

Presently preferred probe-based screening conditions comprise a temperature of about 37° C., a formamide concentration of about 20%, and a salt concentration of about 5× standard saline citrate (SSC; 20×SSC contains 3M sodium chloride, 0.3M sodium citrate, pH 7.0). Such conditions will allow the identification of sequences which have a substantial degree of similarity with the probe sequence, without requiring perfect homology.

“Hybridization” refers to the binding of complementary strands of nucleic acid (i.e., sense:antisense strands or probe:target-DNA) to each other through hydrogen bonds, similar to the bonds that naturally occur in chromosomal DNA. Stringency levels used to hybridize a given probe with target-DNA can be readily varied by those of skill in the art.

The phrase “stringent hybridization conditioned” is used herein to refer to conditions under which polynucleic acid hybrids are stable. As known to those of skill in the art, the stability of hybrids is reflected in the melting temperature (Tₘ) of the hybrids. Tₘ can be approximated by the formula:
81.5° C.−16.6(log₁₀[Na⁺])+0.41(% G+C)−600/1,
where 1 is the length of the hybrids in nucleotides. Tₘ decreases approximately 1°-1.5° C. with every 1% decrease in sequence homology. In general, the stability of a hybrid is a function of sodium ion concentration and temperature. Typically, the hybridization reaction is performed under conditions of lower stringency, followed by washes of varying, but higher, stringency. Reference to hybridization stringency relates to such washing conditions.

As used herein, the phrase “moderately stringent hybridization” refers to conditions that permit target-DNA to bind a complementary nucleic acid that has about 60% identity, preferably about 75% identity, more preferably about 85% identity to the target DNA; with greater than about 90% identity to target-DNA being especially preferred. Preferably, moderately stringent conditions are conditions equivalent to hybridization in 50% formamide, 5× Denhart's solution, 5×SSPE, 0.2% SDS at 42° C., followed by washing in 0.2×SSPE, 0.2% SDS, at 65° C.

The phrase “high stringency hybridization” refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in 50% formamide, 5× Denhart's solution, 5×SSPE, 0.2% SDS at 42° C., followed by washing in 0.1×SSPE, and 0.1% SDS at 65° C.

The phrase “low stringency hybridization” refers to conditions equivalent to hybridization in 10% formamide, 5× Denhart's solution, 6×SSPE, 0.2% SDS at 42° C., followed by washing in 1×SSPE, 0.2% SDS, at 50° C.

Denhardt's solution and SSPE (see, e.g., Sambrook, Fritsch, and Maniatis, in: Molecular Cloning, A Laboratory Manual, Cold Spring Harbor Laboratory Press, 1989) are well known to those of skill in the art as are other suitable hybridization buffers. For example, SSPE is pH 7.4 phosphate-buffered 0.18M NaCl. SSPE can be prepared, for example, as a 20× stock solution by dissolving 175.3 g of NaCl, 27.6 g of NaH₂PO₄ and 7.4 g EDTA in 800 ml of water, adjusting the pH to 7.4, and then adding water to 1 liter. Denhardt's solution (see, Denhardt (1966) Biochem. Biophys. Res. Commun. 23:641) can be prepared, for example, as a 50× stock solution by mixing 5 g Ficoll (Type 400, Pharmacia LKB Biotechnology, INC., Piscataway N.J.), 5 g of polyvinylpyrrolidone, and 5 g bovine serum albumin (Fraction V; Sigma, St. Louis Mo.), and then adding water to 500 ml and filtering to remove particulate matter.

Preferred nucleic acids encoding the invention polypeptide(s) hybridize under moderately stringent, preferably high stringency, conditions to substantially the entire sequence, or substantial portions (i.e., typically at least 15-30 nucleotides) of the nucleic acid sequence set forth in SEQ ID NO:1 (Human GPR54) or 4 (Mouse GPR54).

Preferably, hybridization conditions will be selected which allow the identification of sequences having at least 70% homology with the probe, while discriminating against sequences which have a lower degree of homology with the probe. As a result, nucleic acids having substantially the same nucleotide sequence as the sequence of nucleotides set forth in SEQ ID NO:1 are obtained.

Thus, the nucleic acid probes are useful for various applications. On the one hand, they may be used as PCR primers for amplification of nucleic acid molecules according to the invention. On the other hand, they can be useful tools for the detection of the expression of molecules according to the invention in target tissues, for example, by in-situ hybridization or Northern-Blot hybridization.

The invention probes may be labeled by methods well-known in the art, as described hereinafter, and used in various diagnostic kits.

A “label” refers to a compound or composition that facilitates detection of a compound or composition with which it is specifically associated, which can include conferring a property that makes the labeled compound or composition able to bind specifically to another molecule. “Labeled” refers to a compound or composition that is specifically associated, typically by covalent bonding but non-covalent interactions can also be employed to label a compound or composition, with a label. Thus, a label may be detectable directly, i.e., the label can be a radioisotope (e.g., ³H, ¹⁴C, ³²P, ³⁵S, ¹²⁵I, ¹³¹I) or a fluorescent or phosphorescent molecule (e.g., FITC, rhodamine, lanthanide phosphors), or indirectly, i.e., by enzymatic activity (e.g., horseradish peroxidase, beta-galactosidase, luciferase, alkaline phosphatase) or by its ability to bind to another molecule (e.g., streptavidin, biotin, an antigen, epitope, or antibody). Incorporation of a label can be achieved by a variety of means, i.e., by use of radiolabeled or biotinylated nucleotides in polymerase-mediated primer extension reactions, epitope-tagging via recombinant expression or synthetic means, or binding to an antibody.

Labels can be attached directly or via spacer arms of various lengths, i.e., to reduce steric hindrance. Any of a wide variety of labeled reagents can be used for purposes of the present invention. For instance, one can use one or more labeled nucleoside triphosphates, primers, linkers, or probes. A description of immunofluorescent analytic techniques is found in DeLuca, “Immunofluorescence Analysis”, in Antibody As a Tool, Marchalonis et al., eds., John Wiley & Sons, Ltd., pp. 189-231 (1982), which is incorporated herein by reference.

The term label can also refer to a “tag”, which can bind specifically to a labeled molecule. For instance, one can use biotin as a tag and then use avidinylated or streptavidinylated horseradish peroxidase (HRP) to bind to the tag, and then use a chromogenic substrate (e.g., tetramethylbenzamine) to detect the presence of HRP. In a similar fashion, the tag can be an epitope or antigen (e.g., digoxigenin), and an enzymatically, fluorescently, or radioactively labeled antibody can be used to bind to the tag.

In defining nucleic acid sequences, all subject nucleic acid sequences capable of encoding substantially similar amino acid sequences are considered substantially similar or are considered as comprising substantially identical sequences of nucleotides to the reference nucleic acid sequence, i.e., human GPR54 encoding sequence or the mouse GPR54 encoding sequence.

In practice, the term “substantially the same sequence” means that DNA or RNA encoding two proteins hybridize under moderately stringent conditions and encode proteins that have the same sequence of amino acids or have changes in sequence that do not alter their structure or function.

Nucleotide sequence “similarity” is a measure of the degree to which two polynucleotide sequences have identical nucleotide bases at corresponding positions in their sequence when optimally aligned (with appropriate nucleotide insertions or deletions). Sequence similarity or percent similarity can be determined, for example, by comparing sequence information using sequence analysis software such as the GAP computer program, version 6.0, available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math. 2:482, 1981).

As used herein, “substantially identical sequences of nucleotides” share at least about 90% identity, and substantially identical amino acid sequences share more than 95% amino acid identity. It is recognized, however, that proteins (and DNA or mRNA encoding such proteins) containing less than the above-described level of homology arising as splice variants or that are modified by conservative amino acid substitutions (or substitution of degenerate codons) are contemplated to be within the scope of the present invention.

The present invention also encompasses nucleic acids which differ from the nucleic acids shown in SEQ ID Nos:1 or 4, but which have the same phenotype. Phenotypically similar nucleic acids are also referred to as “functionally equivalent nucleic acids”.

As used herein, the phrase “functionally equivalent nucleic acids” encompasses nucleic acids characterized by slight and non-consequential sequence variations that will function in substantially the same manner to produce the same protein product(s) as the nucleic acids disclosed herein.

Functionally equivalent sequences will function in substantially the same manner to produce substantially the same compositions as the nucleic acid and amino acid compositions disclosed and claimed herein.

In particular, functionally equivalent DNAs encode proteins that are the same as those disclosed herein or that have conservative amino acid variations, such as substitution of a non-polar residue for another non-polar residue or a charged residue for a similarly charged residue. These changes include those recognized by those of skill in the art as those that do not substantially alter the tertiary structure of the protein.

In particular, functionally equivalent nucleic acids encode polypeptides that are the same as those disclosed herein or that have conservative amino acid variations, or that are substantially similar to one having the amino acid sequence as set forth in SEQ. ID. NOs:2 or 5.

In one embodiment of the present invention, cDNAs encoding the invention receptor protein disclosed herein include substantially the same nucleotide sequence as set forth in SEQ ID NO: 1. Preferred cDNA molecules encoding the invention proteins include the same nucleotide sequence as that set forth in SEQ ID NO: 1.

In another embodiment of the present invention, cDNAs encoding the invention receptor protein disclosed herein include substantially the same nucleotide sequence as set forth in SEQ ID NOs: 1 or 4. Preferred cDNA molecules encoding the invention receptor proteins include the same nucleotide sequence as that set forth in SEQ ID NO: 4.

Another embodiment of the invention contemplates nucleic acid(s) having substantially the same nucleotide sequence as the reference nucleotide sequence that encodes substantially the same amino acid sequence as that set forth in SEQ ID NO:2.

Another embodiment of the invention contemplates nucleic acid(s) having substantially the same nucleotide sequence as the reference nucleotide sequence that encodes substantially the same amino acid sequence as that set forth in SEQ ID NO: 5.

Further provided are nucleic acids encoding the invention polypeptides that, by virtue of the degeneracy of the genetic code, do not necessarily hybridize to the invention nucleic acids under specified hybridization conditions. Preferred nucleic acids encoding the invention receptor proteins are comprised of nucleotides that encode substantially the same amino acid sequence set forth in SEQ ID NO: 2 or 5.

As used herein, the term “degenerate” refers to codons that differ in at least one nucleotide from either of SEQ ID NOs:1 or 4, but encode the same amino acids as that set forth in SEQ ID. NO.: 2. (hGPR54) or 5 (mouseGPR54). For example, codons specified by the triplets “UCU”, “UCC”, “UCA”, and “UCG” are degenerate with respect to each other since all four of these codons encode the amino acid serine.

An exemplary nucleic acid encoding human GPR54 receptor protein may be selected from:
(a) DNA encoding the amino acid sequence set forth in SEQ ID NO:2.
(b) DNA that hybridizes to the DNA of (a) under moderately stringent conditions, wherein the DNA encodes biologically active Human GPR54; or
(c) DNA degenerate with respect to either (a) or (b) above, wherein the DNA encodes biologically active Human GPR54.

An exemplary nucleic acid encoding mouse GPR54 receptor protein may be selected from:
(a) DNA encoding the amino acid sequence set forth in SEQ ID NO: 5.
(b) DNA that hybridizes to the DNA of (a) under moderately stringent conditions, wherein the DNA encodes biologically active mouse GPR54; or
(c) DNA degenerate with respect to either (a) or (b) above, wherein the DNA encodes biologically active mouse GPR54.

The invention nucleic acids can be produced by a variety of methods well-known in the art, e.g., the methods described herein, employing PCR amplification using oligonucleotide primers from various regions of SEQ ID NO:1 or 5 and the like.

As used herein, “expression” refers to the process by which polynucleic acids are transcribed into mRNA and translated into peptides, polypeptides, or proteins. If the polynucleic acid is derived from genomic DNA, expression may, if an appropriate eukaryotic host cell or organism is selected, include splicing of the mRNA.

Polynucleotides which are identical or sufficiently identical to a nucleotide sequence contained in SEQ ID NO:1, may be used as hybridization probes for cDNA and genomic DNA or as primers for a nucleic acid amplification (PCR) reaction, to isolate full-length cDNAs and genomic clones encoding polypeptides of the present invention and to isolate cDNA and genomic clones of other genes (including genes encoding homologs and orthologs from species other than human) that have a high sequence similarity to SEQ ID NO:1. Typically these nucleotide sequences are 70% identical, preferably 80% identical, more preferably 90% identical, most preferably 95% identical to that of the referent. The probes or primers will generally comprise at least 15 nucleotides, preferably, at least 30 nucleotides and may have at least 50 nucleotides. Particularly preferred probes will have between 30 and 50 nucleotides.

A polynucleotide encoding a polypeptide of the present invention, including homologs and orthologs from species other than human, may be obtained by a process which comprises the steps of screening an appropriate library under stringent hybridization conditions with a labeled probe having the sequence of SEQ ID NO: 1 or a fragment thereof; and isolating full-length cDNA and genomic clones containing the polynucleotide sequence. Such hybridization techniques are well known to the skilled artisan.

“Polypeptide” or “peptide” or “protein” refers to a polymer of amino acid residues and to variants and synthetic analogs of the same and are used interchangeably herein. Thus, these terms apply to amino acid polymers in which one or more amino acid residues is a synthetic non-naturally occurring amino acid, such as a chemical analog of a corresponding naturally occurring amino acid, as well as to naturally occurring amino acid polymers. The invention receptor protein is the preferred polypeptide.

The term “amino acid sequence” as used herein refers to an oligopeptide, peptide, polypeptide, or protein sequence, and fragments or portions thereof, and to naturally occurring or synthetic molecules.

“Identity,” as known in the art, is a relationship between two or more polypeptide sequences or two or more polynucleotide sequences, as the case may be, as determined by comparing the sequences. In the art, “identity” also means the degree of sequence relatedness between polypeptide or polynucleotide sequences, as the case may be, as determined by the match between strings of such sequences. “Identity” or “homology” with respect to the invention receptor protein is defined herein as the percentage of amino acid residues in the candidate sequence that are identical with the residues in SEQ ID NO: 3, after aligning the sequences and introducing gaps, if necessary, to achieve the maximum percent homology, and not considering any conservative substitutions as part of the sequence identity. No N- nor C-terminal extensions, deletions nor insertions shall be construed as reducing identity or homology.

“Identity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New, York, 1991; and Carillo, H., and Lipman, D., SIAM J Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

Parameters for polypeptide sequence comparison include the following:
1) Algorithm: Needleman and Wunsch, J. Mol. Biol. 48: 443-453 (1970)
Comparison matrix: BLOSSUM62 from Hentikoff and Hentikoff, Proc. Natl. Acad. Sci. USA. 89:10915-10919 (1992)
Gap Penalty: 12
Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Parameters for polynucleotide comparison include the following:
1) Algorithm: Needleman and Wunsch, J. Mol. Biol. 48: 443-453 (1970)
Comparison matrix: matches=+10, mismatch=0
Gap Penalty: 50
Gap Length Penalty: 3

Available as: The “gap” program from Genetics Computer Group, Madison Wis. These are the default parameters for nucleic acid comparisons.

A preferred meaning for “identity” for polynucleotides and polypeptides, as the case may be, are provided in (1) and (2) below.
(1) Polynucleotide embodiments further include an isolated polynucleotide comprising a polynucleotide sequence having at least a 50, 60, 70, 80, 85, 90, 95, 97 or 100% identity to the reference sequence of SEQ ID NO: 1, wherein the polynucleotide sequence may be identical to the reference sequence of SEQ ID NO: 1 or may include up to a certain integer number of nucleotide alterations as compared to the reference sequence, wherein the alterations are selected from the group consisting of at least one nucleotide deletion, substitution, including transition and transversion, or insertion, and wherein the alterations may occur at the 5′ or 3′ terminal positions of the reference nucleotide sequence or anywhere between those terminal positions, interspersed either individually among the nucleotides in the reference sequence or in one or more contiguous groups within the reference sequence, and wherein the number of nucleotide alterations is determined by multiplying the total number of nucleotides in SEQ ID NO: 1 by the integer defining the percent identity divided by 100 and then subtracting that product from the total number of nucleotides in SEQ ID NO: 1, or:
   NₙXₙ−(XₙY),
wherein Nₙ is the number of nucleotide alterations, Xₙ is the total number of nucleotides in SEQ ID NO: 1, Y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and is the symbol for the multiplication operator, and wherein any non-integer product of Xₙ and Y is rounded down to the nearest integer prior to subtracting it from Xₙ Alterations of a polynucleotide sequence encoding the polypeptide of SEQ ID NO:2 may create nonsense, missense or frameshift mutations in this coding sequence and thereby alter the polypeptide encoded by the polynucleotide following such alterations.
(2) Polypeptide embodiments further include an isolated polypeptide comprising a polypeptide having at least a 50, 60, 70, 80, 85, 90, 95, 97 or 100% identity to a polypeptide reference sequence of SEQ ID NO:2, wherein the polypeptide sequence may be identical to the reference sequence of SEQ ID NO: 2 or may include up to a certain integer number of amino acid alterations as compared to the reference sequence, wherein the alterations are selected from the group consisting of at least one amino acid deletion, substitution, including conservative and non-conservative substitution, or insertion, and wherein the alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein the number of amino acid alterations is determined by multiplying the total number of amino acids in SEQ ID NO:2 by the integer defining the percent identity divided by 100 and then subtracting that product from the total number of amino acids in SEQ ID NO:2, or:
   Nₐ=Xₐ−(XₐY),
herein Nₐ is the number of amino acid alterations, Xₐ is the total number of amino acids in SEQ ID NO:2, Y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and is the symbol for the multiplication operator, and wherein any non-integer product of Xₐ and Y is rounded down to the nearest integer prior to subtracting it from Xₐ.

As used herein, a “variant” of the invention receptor protein refers to a polypeptide having an amino acid sequence with one or more amino acid substitutions, insertions, and/or deletions compared to the sequence of the invention receptor protein. Generally, differences are limited so that the sequences of the reference (invention receptor protein) and the variant are closely similar overall, and in many regions, identical. Such variants are generally biologically active and necessarily have less than 100% sequence identity with the polypeptide of interest.

In a preferred embodiment, the biologically active variant has an amino acid sequence sharing at least about 70% amino acid sequence identity with the invention receptor protein, preferably at least about 75%, more preferably at least about 80%, still more preferably at least about 85%, even more preferably at least about 90%, and most preferably at least about 95%. Amino-acid substitutions are preferably substitutions of single amino-acid residues.

A “fragment” of the invention receptor protein (reference protein) is meant to refer to a protein molecule which contains a portion of the complete amino acid sequence of the wild type or reference protein.

Preferred polypeptides and polynucleotides of the present invention are expected to have, inter alia, similar biological functions/properties to their homologous polypeptides and polynucleotides. Furthermore, preferred polypeptides and polynucleotides of the present invention have at least one GPR25 activity.

As used herein, activity of the invention receptor protein refers to any activity characteristic of human GPR54 or its mouse counterpart. Such activity can typically be measured by one or more in vitro methods, and frequently corresponds to an in vivo activity of human or mouse GPR54. Such activity may be measured by any method known to those of skill in the art, such as, for example, assays that measure calcium influx or endogenous cAMP levels.

The invention receptor protein, biologically active fragments, and functional equivalents thereof can also be produced by chemical synthesis. For example, synthetic polypeptides can be produced using Applied Biosystems, Inc. Model 430A or 431A automatic peptide synthesizer (Foster City, Calif.) employing the chemistry provided by the manufacturer.

The present invention also provides compositions containing an acceptable carrier and any of an isolated, purified invention polypeptide, an active fragment thereof, or a purified, mature protein and active fragments thereof, alone or in combination with each other. These polypeptides or proteins can be recombinantly derived, chemically synthesized or purified from native sources.

As used herein, the term “acceptable carrier” encompasses any of the standard pharmaceutical carriers, such as phosphate buffered saline solution, water and emulsions such as an oil/water or water/oil emulsion, and various types of wetting agents.

Also provided are antisense oligonucleotides having a nucleotide sequence capable of binding specifically with any portion of an mRNA that encodes the invention receptor protein so as to prevent translation of the mRNA. The antisense oligonucleotide may have a sequence capable of binding specifically with any portion of the sequence of the cDNA encoding the invention polypeptides.

Invention nucleic acids, oligonucleotides (including antisense), vectors containing same, transformed host cells, polypeptides and combinations thereof, as well as antibodies of the present invention, can be used to screen compounds in vitro to determine whether a compound functions as a potential agonist or antagonist to invention receptor proteins.

These in vitro screening assays provide information regarding the function and activity of invention receptor proteins, which can lead to the identification and design of compounds that are capable of specific interaction with native GPR54 or the human PTH2 receptor.

Accordingly, a method for identifying compounds, which bind to the invention receptor protein(s) are also contemplated by the present invention. The invention receptor protein may be employed in a competitive binding assay. Such an assay can accommodate the rapid screening of a large number of compounds to determine which compounds, if any, are capable of binding to invention receptor protein. Subsequently, more detailed assays can be carried out with those compounds found to bind, to further determine whether such compounds act as modulators, agonists or antagonists of invention receptor protein.

In accordance with another embodiment of the present invention, transformed host cells that recombinantly express the invention receptor protein can be contacted with a test compound, and the modulating effect(s) thereof can then be evaluated by comparing the invention receptor protein-mediated response (e.g., via measurement of second messenger activity/cAMP activity) in the presence and absence of the test compound, or by comparing the response of test cells or control cells, i.e., cells that do not express the invention receptor proteins to the presence of the compound.

As used herein, a compound or a signal that “modulates the activity” of invention receptor protein refers to a compound or a signal that alters the activity of invention receptor protein so that the activity of the invention receptor protein is different in the presence of the compound or signal than in the absence of the compound or signal. In particular, such compounds or signals include agonists and antagonists. Such activity is generally detected by measuring cAMP levels.

The term “agonist” refers to a substance or signal, such as the invention receptor protein, that activates receptor function; and the term “antagonist” refers to a substance that interferes with receptor function. Typically, the effect of an antagonist is observed as a blocking of activation by an agonist. Antagonists include competitive and non-competitive antagonists. A competitive antagonist (or competitive blocker) interacts with or near the site specific for the agonist (e.g., ligand or neurotransmitter) for the same or closely situated site. A non-competitive antagonist or blocker inactivates the functioning of the receptor by interacting with a site other than the site that interacts with the agonist.

As understood by those of skill in the art, assay methods for identifying compounds that modulate invention receptor protein activity generally require comparison to a control. One type of a “control” is a cell or culture that is treated substantially the same as the test cell or test culture exposed to the compound, with the distinction that the “control” cell or culture is not exposed to the compound. For example, in methods that use voltage clamp electrophysiological procedures, the same cell can be tested in the presence or absence of compound, by merely changing the external solution bathing the cell. Another type of “control” cell or culture may be a cell or culture that is identical to the transfected cells, with the exception that the “control” cell or culture do not express the invention receptor protein. Accordingly, the response of the transfected cell to compound is compared to the response (or lack thereof) of the “control” cell or culture to the same compound under the same reaction conditions.

In yet another embodiment of the present invention, the activation of the invention receptor proteins, human GPR544 or mouse GPR54 can be modulated by contacting the receptor proteins with an effective amount of at least one compound (agonist or antagonist) identified by the above-described bioassays.

An alternative method contemplates contacting a cell expressing either one of the human or mouse GPR54 receptor protein with a test compound, and determining the effect of the test compound by measuring level of cAMP as a measure of the modulating effect of the test compound on receptor activity, wherein an increase in cAMP levels is indicative of the modulating effects of the test compound on the receptor protein (agonist), i.e., opening of the receptor protein, while a decrease reflects the opposite (antagonist).

In accordance with another embodiment of the present invention, there are provided methods for diagnosing disease states characterized by abnormal signal transduction. For example, a sample can be obtained from a patient believed to be suffering from a pathological disorder characterized by dysfunctional signal transduction, and contacted with a nucleic acid probe having a sequence of nucleotides that are substantially homologous to the nucleotide sequence set forth in one of SEQ ID NO:1 or 2. Binding of the probe to any complimentary mRNA present in the sample can be determined and is indicative of the regression, progression or onset of such a pathological disorder in the patient.

Alternatively, the patient sample can be contacted with a detectable probe that is specific for the gene product of the invention nucleic acid molecule, under conditions favoring the formation of a probe/gene product complex. The presence of the complex is indicative of the regression, progression or onset of the pathological disorder in the patient.

In accordance with another embodiment of the present invention, there are provided diagnostic systems, preferably in kit form, comprising at least one invention nucleic acid in a suitable packaging material. The diagnostic nucleic acids are derived from the invention receptor protein-encoding nucleic acids described herein. In one embodiment, for example, the diagnostic nucleic acids are derived from SEQ ID NO:1. Invention diagnostic systems are useful for assaying for the presence or absence of nucleic acid encoding the invention receptor protein in either genomic DNA or in transcribed nucleic acid (such as mRNA or cDNA) encoding the invention receptor protein.

A suitable diagnostic system includes at least one invention nucleic acid, preferably two or more invention nucleic acids, as a separately packaged chemical reagent(s) in an amount sufficient for at least one assay. Instructions for use of the packaged reagent are also typically included. Those of skill in the art can readily incorporate invention nucleic probes and/or primers into kit form in combination with appropriate buffers and solutions for the practice of the invention methods as described herein.

“Treatment” refers to both therapeutic treatment and prophylactic or preventative measures. Those in need of treatment include those already with the disorder as well as those prone to have the disorder or those in which the disorder is to be prevented.

A “disorder” is any condition that would benefit from treatment with the invention receptor protein of the invention. This includes chronic and acute disorders or diseases including those pathological conditions which predispose the mammal to the disorder in question. Disorders include, but are not limited to, those of the cardiovascular system, the nervous system and those involving pain perception.

As used herein, “functional” with respect to a recombinant or heterologous human or mouse GPR54 means that the invention receptor proteins exhibits an activity attending native GPR54 as assessed by any in vitro or in vivo assay disclosed herein or known to those of skill in the art. Possession of any such activity that may be assessed by any method known to those of skill in the art and provided herein is sufficient to designate a peptide as functional. Such activity may be detected as noted supra.

In yet another aspect, the screening assays provided by the invention relate to transgenic mammals whose germ cells and somatic cells contain a nucleotide sequence encoding Human or mouse GPR54 protein or a selected portion of the receptor which, e.g., binds ligand, or the like. There are several means by which a sequence encoding, for example, the human GPR54 may be introduced into a non-human mammalian embryo, some of which are described in, e.g., U.S. Pat. No. 4,736,866, Jaenisch, Science 240-1468-1474 (1988) and Westphal et al., Annu. Rev.

Cell Biol. 5:181-196 (1989), which are incorporated herein by reference. The animal's cells then express the receptor and thus may be used as a convenient model for testing or screening selected agonists or antagonists.

Polypeptides of the Invention

The present invention provides isolated nucleic acid molecules that encode a novel human receptor protein. A mouse receptor protein is also provided. Specifically, isolated DNA encoding a human receptor protein—GPR54 are described as are recombinant messenger RNA (mRNA). Splice variants of the isolated DNA are also described. Typically, unless human GPR54 arises as a splice variant, human GPR54-encoding DNA will share substantial sequence homology (i.e., greater than about 90%), with the human GPR54 encoding DNA described herein. DNA or RNA encoding a splice variant may share less than 90% overall sequence homology with the DNA or RNA provided herein, but such a splice variant would include regions of nearly 100% homology to the disclosed DNAs. The same holds true for the mouse GPR54 disclosed herein.

The Human GPR54 receptor proteins of the present invention include the polypeptide of SEQ ID NO:2 (in particular the mature polypeptide) as well as Human GPR54 polypeptides and which have at least 80% identity to the polypeptide of SEQ ID NO:2 or the relevant portion and more preferably at least 85% identity, and still more preferably at least 90% identity, and even still more preferably at least 95% identity to SEQ ID NO: 2.

The Human GPR54 receptor proteins may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

Biologically active fragments of the Human 0GPR54 polypeptides are also included in the invention. A fragment is a polypeptide having an amino acid sequence that entirely is the same as part, but not all, of the amino acid sequence of the aforementioned Human GPR54 polypeptides.

As with Human GPR54 receptor proteins, fragments may be “free-standing,” or comprised within a larger polypeptide of which they form a part or region, most preferably as a single continuous region.

Preferred fragments include, for example, truncation polypeptides having the amino acid sequence of Human GPR54 receptor proteins, except for deletion of a continuous series of residues that includes the amino terminus, or a continuous series of residues that includes the carboxyl terminus or deletion of two continuous series of residues, one including the amino terminus and one including the carboxyl terminus.

Biologically active fragments are those that mediate receptor activity, including those with a similar activity or an improved activity, or with a decreased undesirable activity. Also included are those that are antigenic or immunogenic in an animal, especially in a human.

Thus, the receptor proteins of the invention include polypeptides having an amino acid sequence at least 80% identical to that of SEQ ID NO:2 or fragments thereof with at least 85% identity to the corresponding fragment of SEQ ID NO:2.

Preferably, all of these polypeptides retain the biological activity of the receptor protein disclosed herein, including antigenic activity. Included in this group are variants of the defined sequence and fragments. Preferred variants are those that vary from the referents by conservative amino acid substitutions—i.e., those that substitute a residue with another of like characteristics. Typical such substitutions are among Ala, Val, Leu and lie; among Ser and Thr; among the acidic residues Asp and Glu; among Asn and Gin; and among the basic residues Lys and Arg; or aromatic residues Phe and Tyr. Particularly preferred are variants in which several, 5-10, 1-5, or 1-2 amino acids are substituted, deleted, or added in any combination.

The Human GPR54 polypeptides of the invention can be prepared in any suitable manner. Such polypeptides include isolated naturally occurring polypeptides, recombinantly produced polypeptides, synthetically produced polypeptides, or polypeptides produced by a combination of these methods.

Recombinant polypeptides of the present invention may be prepared by processes well known in the art from genetically engineered host cells comprising expression systems. Accordingly, in a further aspect, the present invention relates to expression systems which comprise a polynucleotide or polynucleotides of the present invention, to host cells which are genetically engineered with such expression systems and to the production of polypeptides of the invention by recombinant techniques. Cell-free translation systems can also be employed to produce such proteins using RNAs derived from the DNA constructs of the present invention.

Polynucleotides of the Invention

Another aspect of the invention relates to isolated polynucleotides which encode the Human GPR54 receptor proteins and polynucleotides closely related thereto.

Human GPR54 receptor protein of the invention is a member of G-Protein coupled receptor superfamily. The cDNA sequence contains an open reading frame encoding a protein of 398 with a deduced molecular weight of ca 42.6 kDa.

Complementary DNA clones encoding the invention peptide may be prepared from the DNA provided. As well, the polynucleotides of the invention can be obtained from natural sources such as genomic DNA libraries or can be synthesized using well known and commercially available techniques.

Indeed, in one aspect, the polynucleotide of the present invention encoding a Human GPR54 receptor protein may be obtained using standard cloning and screening, from a cDNA library derived from mRNA in cells of human hypothalamus using the expressed sequence tag (EST) analysis (Adams, M. D., et al. Science (1991) 252:1651-1656; Adams, M. D. et al., Nature, (1992) 355:632-634; Adams, M. D., et al., Nature (1995) 377 Supp:3-174).

The nucleotide sequence encoding Human GPR54 receptor protein may be identical over its entire length to the coding sequence set forth in one of SEQ ID NO:1, or may be a degenerate form of this nucleotide sequence encoding the polypeptide of SEQ ID NO:2, or may be highly identical to a nucleotide sequence that encodes the polypeptide of SEQ ID NO:2.

Preferably, the nucleic acid molecules of the invention, i.e., SEQ ID NO: 1 contain a nucleotide sequence that is highly identical, at least 80% identical, with a nucleotide sequence encoding a Human GPR54 receptor protein, or at least 85% identical with the encoding nucleotide sequence set forth in SEQ ID NO:1, or at least 90% identical to a nucleotide sequence encoding the polypeptide of SEQ ID NO:2.

Among particularly preferred embodiments of the invention are polynucleotides encoding Human GPR54 receptor proteins having the amino acid sequence of set out in SEQ ID NO:2 and variants thereof.

Further preferred embodiments are polynucleotides encoding Human GPR54 receptor protein variants that have the amino acid sequence of the Human GPR54 of SEQ ID NO:2 in which several, 5-10, 1-5,1-3, 1-2 or 1 amino acid residues are substituted, deleted or added, in any combination.

Further preferred embodiments of the invention are polynucleotides that are at least 80% identical over their entire length to a polynucleotide encoding the Human GPR54 receptor protein having the amino acid sequence set out in SEQ ID NO:2, and polynucleotides which are complementary to such polynucleotides. In this regard, polynucleotides at least 80% identical over their entire length to the same are particularly preferred, and those with at least 90% are especially preferred. Furthermore, those with at least 97% are highly preferred and those with at least 98-99% are most highly preferred, with at least 99% being the most preferred.

The present invention further relates to polynucleotides that hybridize to the herein above-described sequences. In this regard, the present invention especially relates to polynucleotides which hybridize under stringent conditions to the herein above-described polynucleotides. As herein used, the term “stringent conditions” means hybridization will occur only if there are at least 95% and preferably at least 97% identity between the sequences.

The invention nucleotide sequences were isolated employing analogous rat DNA encoding rat GPR54. In addition to their use as coding sequences for the production of Human GPR54 receptor proteins and synthetic human receptors, the invention polynucleotide sequences can also be used as probes for the identification of additional human GPR54 receptor protein sequences.

As noted, polynucleotides of the invention, which are sufficiently identical to a nucleotide sequence contained in SEQ ID NO:1, may be used as hybridization probes for cDNA and genomic DNA, to isolate full-length cDNAs and genomic clones encoding Human GPR54 and to isolate cDNA and genomic clones of other genes that have a high sequence similarity to the Human GPR54 encoding gene. Such hybridization techniques are known to those of skill in the art. Typically these nucleotide sequences are 70% identical, preferably 80% identical, more preferably 90% identical to that of the referent.

Nucleic acid probes derived from the invention polynucleotide sequences are particularly useful for this purpose. Examples of nucleic acids are RNA, cDNA, or isolated genomic DNA encoding the invention receptor protein. Such nucleic acids may include, but are not limited to, nucleic acids having substantially the same nucleotide sequence as set forth in SEQ ID NO: 1 or SEQ ID NO: 4 or one encoding the amino acid sequence as set forth in SEQ ID NO:2 or 5. The probes generally will comprise at least 15 nucleotides. Preferably, such probes will have at least 30 nucleotides and may have at least 50 nucleotides. Particularly preferred probes will range between 30 and 50 nucleotides. The probe may be used to isolate splice variants of the polynucleotides disclosed herein.

Thus, one means of isolating a nucleic acid encoding Human GPR54 receptor protein is to probe various sources of human hypothalamic cDNA with the invention sequences, and then select those sequences having a significant level of sequence homology with the probe employed. Generally, after screening the mammalian library, positive clones are identified by detecting a hybridization signal; the identified clones are characterized by restriction enzyme mapping and/or DNA sequence analysis, and then examined, by comparison with the sequences set forth herein, to ascertain whether they include DNA encoding the entire invention receptor protein. If the selected clones are incomplete, they may be used to rescreen the same or a different library to obtain overlapping clones. If desired, the library can be rescreened with positive clones until overlapping clones that encode an entire invention receptor protein are obtained. If the library is a cDNA library, then the overlapping clones will include an open reading frame. If the library is genomic, then the overlapping clones may include exons and introns. In both instances, complete clones may be identified by comparison with the DNA and encoded proteins provided herein.

Preferred stringent hybridization conditions include overnight incubation at 42.degree. C. in a solution comprising: 50% formamide, 5×SSC (150 mM NaCl, 15 mM trisodium citrate), 50 mM sodium phosphate (pH7.6), 5× Denhardt's solution, 10% dextran sulfate, and 20 microgram/ml denatured, sheared salmon sperm DNA; followed by washing the filters in 0.1×SSC at about 65.degree. C. Thus the present invention also includes polynucleotides obtainable by screening an appropriate library under stringent hybridization conditions with a labeled probe having the sequence of SEQ ID NO: 1 or a fragment thereof.

The skilled artisan will appreciate that, in many cases, an isolated cDNA sequence will be incomplete, in that the region coding for the human receptor protein of the invention is cut short at the 5′ end of the cDNA. This is a consequence of reverse transcriptase, an enzyme with inherently low ‘processivity’ (a measure of the ability of the enzyme to remain attached to the template during the polymerization reaction), failing to complete a DNA copy of the mRNA template during 1st strand cDNA synthesis.

There are several methods available and well known to those skilled in the art to obtain full-length cDNAs, or extend short cDNAs, for example those based on the method of Rapid Amplification of cDNA ends (RACE) (see, for example, Frohman et al., PNAS USA 85, 8998-9002, 1988). Recent modifications of the technique, exemplified by the Marathon™ technology (Clontech Laboratories Inc.) for example, have significantly simplified the search for longer cDNAs. In the Marathon™ technology, cDNAs have been prepared from mRNA extracted from a chosen tissue and an ‘adaptor’ sequence ligated onto each end. Nucleic acid amplification (PCR) is then carried out to amplify the ‘missing’ 5′ end of the cDNA using a combination of gene specific and adaptor specific oligonucleotide primers. The PCR reaction is then repeated using ‘nested’ primers, that is, primers designed to anneal within the amplified product (typically an adaptor specific primer that anneals further 3′ in the adaptor sequence and a gene specific primer that anneals further 5′ in the known gene sequence). The products of this reaction can then be analyzed by DNA sequencing and a full-length cDNA constructed either by joining the product directly to the existing cDNA to give a complete sequence, or carrying out a separate full-length PCR using the new sequence information for the design of the 5′ primer.

Invention DNA sequences or cDNA sequences thus identified can be used for producing invention receptor proteins, when such nucleic acids are incorporated into a variety of protein expression systems known to those of skill in the art. In addition, such nucleic acid molecules or fragments thereof can be labeled with a readily detectable substituent and used as hybridization probes for assaying for the presence and/or amount of a Human GPR54 encoding gene or mRNA transcript in a given sample. The nucleic acid molecules described herein, and fragments thereof, are also useful as primers and/or templates in a PCR reaction for amplifying genes encoding the invention protein described herein.

In accordance with the above, host cells are transfected with DNA encoding the invention receptor protein. Using methods such as northern blot or slot blot analysis, transfected cells that contain invention receptor protein encoding DNA or RNA can be selected. Transfected cells can also be analyzed to identify those that express the invention receptor protein. Analysis can be carried out, for example, by using any of well known screening assays attending a functional receptor, and comparing the values obtained to a control, untransfected host cells by electrophysiologically monitoring the currents through the cell membrane in response to invention receptor protein, and the like.

Nucleic acid molecules may be stably incorporated into cells or may be transiently introduced using methods known in the art. Stably transfected mammalian cells may be prepared by transfecting cells with an expression vector comprising a sequence of nucleotides that encodes the invention receptor proteins, i.e., either Human GPR54 or Mouse GPR54, in conjunction with a selectable marker gene (such as, for example, the gene for thymidine kinase, dihydrofolate reductase, neomycin resistance, and the like), and growing the transfected cells under conditions selective for cells expressing the marker gene. To prepare transient transfectants, mammalian cells are transfected with a reporter gene (such as the E. coli .beta.-galactosidase gene) to monitor transfection efficiency. The precise amounts and ratios of DNA encoding the invention receptor proteins may be empirically determined and optimized for a particular cells and assay conditions. Selectable marker genes are typically not included in the transient transfections because the transfectants are typically not grown under selective conditions, and are usually analyzed within a few days after transfection.

Cloned DNA sequences may be introduced into cultured mammalian cells by, for example, calcium phosphate-mediated transfection (Wigler et al., Cell 14: 725, 1978; Corsaro and Pearson, Somatic Cell Genetics 7: 603, 1981; Graham and Van der Eb, Virology 52: 456, 1973.) Other techniques for introducing cloned DNA sequences into mammalian cells, such as electroporation (Neumann et al., EMBO J. 1: 841-845, 1982), may also be used. In order to identify cells that have integrated the cloned DNA, a selectable marker is generally introduced into the cells along with the gene or cDNA of interest. Preferred selectable markers for use in cultured mammalian cells include genes that confer resistance to drugs, such as neomycin, hygromycin, and methotrexate. The selectable marker may be an amplifiable selectable marker. Preferred amplifiable selectable markers are the DHFR gene and the neomycin resistance gene. Selectable markers are reviewed by Thilly (Mammalian Cell Technology, Butterworth Publishers, Stoneham, Mass., which is incorporated herein by reference). The choice of selectable markers is well within the level of ordinary skill in the art.

Selectable markers may be introduced into the cell on a separate plasmid at the same time as the gene of interest, or they may be introduced on the same plasmid. If on the same plasmid, the selectable marker and the gene of interest may be under the control of different promoters or the same promoter, the latter arrangement producing a dicistronic message. Constructs of this type are known in the art (for example, Levinson and Simonsen, U.S. Pat. No. 4,713,339). It may also be advantageous to add additional DNA, known as “carrier DNA” to the mixture which is introduced into the cells.

Host cells containing DNA constructs of the present invention are then cultured to produce recombinant Human GPR54 receptor proteins. Drug selection is then applied to select for growth of cells that are expressing the selectable marker in a stable fashion. Transfected cells may also be selected in the presence of antagonist to inhibit the activity of the receptor. For cells that have been transfected with an amplifiable selectable marker the drug concentration may be increased in a stepwise manner to select for increased copy number of the cloned sequences, thereby increasing expression levels. The cells are cultured according to accepted methods in a culture medium containing nutrients required for growth of mammalian or other host cells. A variety of suitable media are known in the art and generally include a carbon source, a nitrogen source, essential amino acids, vitamins, minerals and growth factors. The growth medium will generally select for cells containing the DNA construct by, for example, drug selection or deficiency in an essential nutrient which is complemented by the selectable marker on the DNA construct or co-transfected with the DNA construct.

Similarly, a variety of suitable yeast cells are readily available to host cells for the invention sequences. Especially preferred are yeast selected from Pichia pastoris, Saccharomyces cerevisiae, Candida tropicalis, Hansenula polymorpha, and the like.

In particularly preferred aspects, eukaryotic cells which contain heterologous DNAs express such DNA and form recombinant invention receptor protein. In more preferred aspects, recombinant invention receptor protein activity is readily detectable because it is a type that is absent from the untransfected host cell.

Heterologous DNA may be maintained in the cell as an episomal element or may be integrated into chromosomal DNA of the cell. The resulting recombinant cells may then be cultured or subcultured (or passaged, in the case of mammalian cells) from such a culture or a subculture thereof. Methods for transfection, injection and culturing recombinant cells are known to the skilled artisan. Similarly, the invention receptor protein(s) may be purified using protein purification methods known to those of skill in the art. For example, antibodies or other ligands that specifically bind to Human GPR54 may be used for affinity purification of the invention receptor protein.

As used herein, “heterologous or foreign DNA and/or RNA” are used interchangeably and refer to DNA or RNA that does not occur naturally as part of the genome of the cell in which it is present or to DNA or RNA which is found in a location or locations in the genome that differ from that in which it occurs in nature. Typically, heterologous or foreign DNA and RNA refers to DNA or RNA that is not endogenous to the host cell and has been artificially introduced into the cell. Examples of heterologous DNA include DNA that encodes the invention receptor proteins.

In preferred embodiments, DNA is ligated into a vector, and introduced into suitable host cells to produce transformed cell lines that express the invention receptor protein, or a fragment thereof. The resulting cell lines can then be produced in quantity for reproducible quantitative analysis of the effects of drugs on receptor function.

In other embodiments, mRNA may be produced by in vitro transcription of DNA encoding the invention receptor protein. This mRNA can then be injected into Xenopus oocytes where the RNA directs the synthesis of the invention receptor protein. Alternatively, the invention-encoding DNA can be directly injected into oocytes for expression of a functional invention receptor protein. The transfected mammalian cells or injected oocytes may then be used in the methods of drug screening provided herein.

Eukaryotic cells in which DNA or RNA may be introduced include any cells that are transfectable by such DNA or RNA or into which such DNA or RNA may be injected. Preferred cells are those that can be transiently or stably transfected and also express the DNA and RNA. Presently most preferred cells are those that can express recombinant or heterologous Human GPR54 encoded by the heterologous DNA. Such cells may be identified empirically or selected from among those known to be readily transfected or injected.

Exemplary cells for introducing DNA include cells of mammalian origin (e.g., COS cells, mouse L cells, Chinese hamster ovary (CHO) cells, human embryonic kidney cells, African green monkey cells and other such cells known to those of skill in the art), amphibian cells (e.g., Xenopus laevis oocytes), yeast cells (e.g., Saccharomyces cerevisiae, Pichia pastons), and the like. Exemplary cells for expressing injected RNA transcripts include Xenopus laevis oocytes. Cells that are preferred for transfection of DNA are known to those of skill in the art or may be empirically identified, and include HEK 293; Ltk⁻ cells; COS-7 cells; and DG44 cells (dhrf CHO cells; see, e.g., Urlaub et al. (1986) Cell. Molec. Genet. 12:555). Other mammalian expression systems, including commercially available systems and other such systems known to those of skill in the art, for expression of DNA encoding the invention receptor protein provided herein are presently preferred.

Alternatively, the invention DNA sequences can be transcribed into RNA, which can then be transfected into amphibian cells for translation into protein. Suitable amphibian cells include Xenopus oocytes.

Vectors, Host Cells, Expression etc.

The present invention also relates to vectors which comprise a polynucleotide or polynucleotides of the present invention, and host cells which are genetically engineered with vectors of the invention and to the production of polypeptides of the invention by recombinant techniques. Cell-free translation systems can also be employed to produce such proteins using RNAs derived from the DNA constructs of the present invention.

Expression vectors for use in carrying out the present invention will comprise a promoter capable of directing the transcription of a cloned DNA and a transcriptional terminator.

An example of the means for preparing the invention receptor protein(s) is to express nucleic acids encoding the invention receptor proteins in a suitable host cell, such as a bacterial cell, a yeast cell, an amphibian cell (i.e., oocyte), or a mammalian cell, using methods well known in the art, and recovering the expressed polypeptide, again using well-known methods. Invention receptor protein(s) can be isolated directly from cells that have been transformed with expression vectors comprising nucleic acid encoding the invention receptor proteins or fragments/portions thereof.

Incorporation of cloned DNA into a suitable expression vector, transfection of eukaryotic cells with a plasmid vector or a combination of plasmid vectors, each encoding one or more distinct genes or with linear DNA, and selection of transfected cells are well known in the art (see, e.g., Sambrook et al. (1989) Molecular Cloning: A Laboratory Manual, Second Edition, Cold Spring Harbor Laboratory Press). Suitable means for introducing (transducing) expression vectors containing invention nucleic acid constructs into host cells to produce transduced recombinant cells (i.e., cells containing recombinant heterologous nucleic acid) are well-known in the art (see, for review, Friedmann, 1989, Science, 244:1275-1281; Mulligan, 1993, Science, 260:926-932, each of which are incorporated herein by reference in their entirety).

Exemplary methods of transduction include, e.g., infection employing viral vectors (see, e.g., U.S. Pat. Nos. 4,405,712 and 4,650,764), calcium phosphate transfection (U.S. Pat. Nos. 4,399,216 and 4,634,665), dextran sulfate transfection, electroporation, lipofection (see, e.g., U.S. Pat. Nos. 4,394,448 and 4,619,794), cytofection, particle bead bombardment, and the like. The heterologous nucleic acid can optionally include sequences which allow for its extrachromosomal (i.e., episomal) maintenance, or the heterologous nucleic acid can be donor nucleic acid that integrates into the genome of the host. Recombinant cells can then be cultured under conditions whereby the invention receptor protein(s) encoded by the DNA is (are) expressed. Preferred cells include mammalian cells (e.g., HEK 293, CHO and Ltk⁻ cells), yeast cells (e.g., methylotrophic yeast cells, such as Pichia pastoris), bacterial cells (e.g., Escherichia coli), and the like.

Suitable expression vectors are well-known in the art, and include vectors capable of expressing DNA operatively linked to a regulatory sequence, such as a promoter region that is capable of regulating expression of such DNA. Thus, an expression vector refers to a recombinant DNA or RNA construct, such as a plasmid, a phage, recombinant virus or other vector that, upon introduction into an appropriate host cell, results in expression of the inserted DNA. Appropriate expression vectors are well known to those of skill in the art and include those that are replicable in eukaryotic cells and/or prokaryotic cells and those that remain episomal or those which integrate into the host cell genome.

Exemplary expression vectors for transformation of E. coli prokaryotic cells include the pET expression vectors (Novagen, Madison, Wis., see U.S. Pat. No. 4,952,496), e.g., pETlla, which contains the T7 promoter, T7 terminator, the inducible E. coli lac operator, and the lac repressor gene; and pET 12a-c, which contains the T7 promoter, T7 terminator, and the E. coli ompT secretion signal. Another such vector is the pIN-IIIompA2 (see Duffaud et al., Meth. in Enzymology, 153:492-507, 1987), which contains the lpp promoter, the lacUV5 promoter operator, the ompA secretion signal, and the lac repressor gene.

Exemplary eukaryotic expression vectors include eukaryotic cassettes, such as the pSV-2 gpt system (Mulligan et al., 1979, Nature, 277:108-114); the Okayama-Berg system (Mol. Cell Biol., 2:161-170), and the expression cloning vector described by Genetics Institute (1985, Science, 228:810-815). Each of these plasmid vectors is capable of promoting expression of the invention chimeric protein of interest.

Representative examples of appropriate host cells for use in practicing the present invention include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

Fungal cells, including species of yeast (e.g., Saccharomyces spp., particularly S. cerevisiae, Schizosaccharomyces spp.) or filamentous fungi (e.g., Aspergillus spp., Neurospora spp.) may be used as host cells within the present invention. Suitable yeast vectors for use in the present invention include YRp7 (Struhl et al., Proc. Natl.

Acad. Sci. USA. 76: 1035-1039, 1978), YEp13 (Broach et al., Gene 8: 121-133, 1979), POT vectors (Kawasaki et al, U.S. Pat. No. 4,931,373, which is incorporated by reference herein), pJDB249 and pJDB219 (Beggs, Nature 275:104-108, 1978) and derivatives thereof. Such vectors will generally include a selectable marker, which may be one of any number of genes that exhibit a dominant phenotype for which a phenotypic assay exists to enable transformants to be selected. Preferred selectable markers are those that complement host cell auxotrophy, provide antibiotic resistance or enable a cell to utilize specific carbon sources, and include LEU2 (Broach et al., ibid.), URA3 (Botstein et al., Gene 8: 17, 1979), HIS3 (Struhl et al., ibid.) or POT1 (Kawasaki et al., ibid.). Another suitable selectable marker is the CAT gene, which confers chloramphenicol resistance on yeast cells.

A variety of higher eukaryotic cells may serve as host cells for expression of the receptor proteins of the invention, although not all cell lines will be capable of functional coupling of the receptor to the cell's second messenger systems. Cultured mammalian cells, such as BHK, CH0, Y1 (Shapiro et al., TIPS Suppl. 4346 (1989)), NG108-15 (Dawson et al., Neuroscience Approached Through Cell Culture, Vol. 2, pages 89-114 (1989)), N1E-115 (Liles et al., J. Biol. Chem. 261:5307-5313 (1986)), PC 12 and COS-1 (ATCC CRL 1650) are preferred. Preferred BHK cell lines are the tk.sup.—ts13 BHK cell line (Waechter and Baserga, Proc. Natl. Acad. Sci. USA 79:1106-1110 (1982)) and the BHK 570 cell line (deposited with the American Type Culture Collection, 12301 Parklawn Dr., Rockville, Md. under accession number CRL 10314). A tk.sup.—BHK cell line is available from the ATCC under accession number CRL 1632.

A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector which is able to maintain, propagate or express a polynucleotide to produce a polypeptide in a host may be used. The appropriate nucleotide sequence may be inserted into an expression system by any of a variety of well-known and routine techniques, such as, for example, those set forth in Sambrook et al., MOLECULAR CLONING, A LABORATORY MANUAL (supra).

Also contained in the expression vectors is a polyadenylation signal located downstream of the coding sequence of interest. Polyadenylation signals include the early or late polyadenylation signals from SV40 (Kaufman and Sharp, ibid.), the polyadenylation signal from the Adenovirus 5 E1B region and the human growth hormone gene terminator (DeNoto et al., Nuc. Acid Res. 9: 3719-3730, 1981). The expression vectors may include a noncoding viral leader sequence, such as the Adenovirus 2 tripartite leader, located between the promoter and the RNA splice sites. Preferred vectors may also include enhancer sequences, such as the SV40 enhancer and the mouse .mu. enhancer (Gillies, Cell 33: 717-728, 1983). Expression vectors may also include sequences encoding the adenovirus VA RNAs.

Additional vectors, promoters and terminators for use in expressing the receptor of the invention in yeast are well known in the art and are reviewed by, for example, Emr, Meth. Enzymol. 185:231-279, (1990), incorporated herein by reference. The receptors of the invention may be expressed in Aspergillus spp. (McKnight and Upshall, described in U.S. Pat. No. 4,935,349, which is incorporated herein by reference). Useful promoters include those derived from Aspergillus nidulans glycolytic genes, such as the ADH3 promoter (McKnight et al., EMBO J. 4:2093-2099, 1985) and the tpiA promoter. An example of a suitable terminator is the ADH3 terminator (McKnight et al., ibid.). Techniques for transforming fungi are well known in the literature, and have been described, for instance by Beggs (ibid.), Hinnen et al. (Proc.

Natl. Acad. Sci. USA 75:1929-1933, 1978), Yelton et al. (Proc. Natl. Acad. Sci. USA 81:1740-1747, 1984), and Russell (Nature 301:167-169, 1983) each of which are incorporated herein by reference.

For secretion of the translated protein into the lumen of the endoplasmic reticulam, into the periplasmic space or into the extracellular environment, appropriate secretion signals may be incorporated into the desired polypeptide. The signal sequence may be derived from the GPR54 coding sequence(s), from other signal sequences described in the art, or synthesized de novo.

If a receptor protein of the present invention is to be expressed for use in screening assays, it is generally preferred that the protein be produced at the surface of the cell.

In this event, the cells may be harvested prior to use in the screening assay. If the receptor protein is secreted into the medium, the medium can be recovered in order to recover and purify the receptor protein. If, on the other hand, it is produced intracellularly, the cells must first be lysed before the receptor protein is recovered.

The receptor proteins of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during isolation and or purification. Methods of protein purification are known in the art (see generally, Scopes, R., Protein Purification, springer-Verlag, N.Y. (1982), which is incorporated herein by reference) and may be applied to the purification of the GPR54 receptor protein and particularly the recombinantly produced GPR54 receptor protein described herein.

In another method of purification, the recombinant gene encoding the receptor proteins of the invention or portions thereof can be modified at the amino terminus, just behind a signal peptide, with a sequence coding for a small hydrophilic peptide, such as described in U.S. Pat. Nos. 4,703,004 and 4,782,137, incorporated herein by reference. Specific antibodies for the peptide facilitate rapid purification of Glu.sub.G R.sub., and the short peptide can then be removed with enterokinase.

Diagnostic Assays

This invention also relates to the use of human GPR54 encoding polynucleotides for use as diagnostic reagents. Detection of a mutated form of Human GPR54 gene associated with a dysfunction will provide a diagnostic tool that can add to or define a diagnosis of a disease or susceptibility to a disease which results from under-expression, over-expression or altered expression of Human GPR54. Individuals carrying mutations in the Human GPR54 gene may be detected at the DNA level by a variety of techniques.

Nucleic acids for diagnosis may be obtained from a subject's cells, such as from blood, urine, saliva, tissue biopsy or autopsy material. The genomic DNA may be used directly for detection. For example, the Human GPR54 DNA may be directly detected in cells with a labeled Human oGPR54 DNA or synthetic oligonucleotide probe in a hybridization procedure similar to the Southern or dot blot. Alternatively, also, the polymerase chain reaction (Saiki et al., Science 239:487 (1988), and U.S. Pat. No. 4,683,195) may be used to amplify DNA sequences, which are subsequently detected by their characteristic size on agarose gels, Southern blot of these gels using Glu _{GR} DNA or a oligonucleotide probe, or a dot blot using similar probes. The probes may comprise from about 14 nucleotides to about 25 or more nucleotides, preferably, 40 to 60 nucleotides, and in some instances a substantial portion or even the entire cDNA of Glu _{GR} R may be used. The probes are labeled with a detectable signal, such as an enzyme, biotin, a radionuclide, fluorophore, chemiluminescer, paramagnetic particle, etc. RNA or cDNA may also be used in similar fashion.

Deletions and insertions can be detected by a change in size of the amplified product in comparison to the normal genotype. Point mutations can be identified by hybridizing amplified DNA to labeled Human GPR54 nucleotide sequences. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, e.g., Myers et al., Science (1985) 230:1242. Sequence changes at specific locations may also be revealed by nuclease protection assays, such as RNase and S1 protection or the chemical cleavage method. See Cotton et al., Proc Natl Acad Sci USA (1985) 85: 4397-4401.

The diagnostic assays offer a process for diagnosing or determining a susceptibility to infections such as bacterial, fungal, protozoan and viral infections, pain; cancers; anorexia; bulimia; asthma; Parkinson's disease; acute heart failure; hypotension; hypertension; urinary retention; osteoporosis; angina pectoris; myocardial infarction; ulcers; asthma; allergies; benign prostatic hypertrophy; and psychotic and neurological disorders, including anxiety, schizophrenia, manic depression, delirium, dementia, severe mental retardation and dyskinesias, such as Huntington's disease or Gilles dela Tourett's syndrome through detection of mutation in the Human GPR54 gene by the methods described.

Additional GPR54 related diseases or pathological condition's associated with its under- or over-expression can be diagnosed by methods comprising determining from a sample derived from a subject an abnormally decreased or increased level of Human GPR54 receptor protein receptor or Human GPR54 mRNA. Decreased or increased expression can be measured at the RNA level using any of the methods well known in the art for the quantitation of polynucleotides, such as, for example, PCR, RT-PCR, RNase protection, Northern blotting and other hybridization methods. Assay techniques that can be used to determine levels of a protein, such as a Human GPR54, in a sample derived from a host are well-known to those of skill in the art.

Such assay methods include radioimmunoassays, competitive-binding assays, Western Blot analysis and ELISA assays.

Chromosome Assays

The nucleic acid molecules of the present invention are also valuable for chromosome identification. The sequence is specifically targeted to and can hybridize with a particular location on an individual human chromosome. The mapping of relevant sequences to chromosomes according to the present invention is an important first step in correlating those sequences with gene associated disease. Once a sequence has been mapped to a precise chromosomal location, the physical position of the sequence on the chromosome can be correlated with genetic map data. Such data are found, for example, in V. McKusick, Mendelian Inheritance in Man (available on line through Johns Hopkins University Welch Medical Library). The relationship between genes and diseases that have been mapped to the same chromosomal region are then identified through linkage analysis (coinheritance of physically adjacent genes). The differences in the cDNA or genomic sequence between affected and unaffected individuals can also be determined. If a mutation is observed in some or all of the affected individuals but not in any normal individuals, then the mutation is likely to be the causative agent of the disease.

Antibodies

The proteins of the invention or their fragments or analogs thereof, or cells expressing them can also be used as immunogens to produce antibodies immunospecific for the Human GPR54 polypeptides.

The term “immunospecific” means that the antibodies have substantial greater affinity for the polypeptides of the invention than their affinity for other related polypeptides in the prior art.

“Immunologically active fragment(s)” of the invention receptor proteins are also embraced by the invention. Such fragments are those proteins that are capable of raising Human GPR54-specific antibodies in a target immune system (e.g., murine or rabbit) or of competing with native Human GPR54 for binding to human GPR54-specific antibodies, and is thus useful in immunoassays for the presence of Human GPR54 peptides in a biological sample. Such immunologically active fragments typically have a minimum size of 8 to 11 consecutive amino acids of a native Human GPR54 peptide.

For example, polyclonal and monoclonal antibodies can be produced by methods well known in the art, as described, for example, in Harlow and Lane, Antibodies: A Laboratory Manual (Cold Spring Harbor Laboratory (1988)), which is incorporated herein by reference. Invention polypeptides can be used as immunogens in generating such antibodies. Alternatively, synthetic peptides can be prepared (using commercially available synthesizers) and used as immunogens. Amino acid sequences can be analyzed by methods well known in the art to determine whether they encode hydrophobic or hydrophilic domains of the corresponding polypeptide. Altered antibodies such as chimeric, humanized, CDR-grafted or bifunctional antibodies can also be produced by methods well known in the art. Such antibodies can also be produced by hybridoma, chemical synthesis or recombinant methods described, for example, in Sambrook et al., supra., and Harlow and Lane, supra. Both anti-peptide and anti-fusion protein antibodies can be used. (see, for example, Bahouth et al., Trends Pharmacol. Sci. 12:338 (1991); Ausubel et al., Current Protocols in Molecular Biology (John Wiley and Sons, N.Y. (1989) which are incorporated herein by reference).

For preparation of monoclonal antibodies, any technique which provides antibodies produced by continuous cell line cultures can be used. Examples include the hybridoma technique (Kohler, G. and Milstein, C., Nature (1975) 256:495497), the trioma technique, the human B-cell hybridoma technique (Kozbor et al., Immunology Today (1983) 4:72) and the EBV-hybridoma technique (Cole et al, MONOCLONAL ANTIBODIES AND CANCER THERAPY, pp. 77-96, Alan R. Liss, Inc., 1985). Active fragments of antibodies are encompassed within the definition of “antibody”.

As the generation of human monoclonal antibodies to either the human or mouse GPR54 antigen may be difficult with conventional techniques, it may be desirable to transfer antigen binding regions of the non-human antibodies, e.g. the F(ab).sub.2 or hypervariable regions, to human constant regions (Fc) or framework regions by recombinant DNA techniques to produce substantially human molecules. Such methods are generally known in the art and are described in, for example, U.S. Pat. No. 4,816,397, EP publications 173,494 and 239,400, which are incorporated herein by reference. Alternatively, one may isolate DNA sequences which code for a human monoclonal antibody or portions thereof that specifically bind to the human receptor protein by screening a DNA library from human B cells according to the general protocol outlined by Huse et al., Science 246:1275-1281 (1989), incorporated herein by reference, and then cloning and amplifying the sequences which encode the antibody (or binding fragment) of the desired specificity.

The antibodies preferably substantially human to minimize immunogenicity and are in substantially pure form. By substantially human is meant generally containing at least about 70% human antibody sequence, preferably at least about 80% human, and most preferably at least about 90-95% or more of a human antibody sequence to minimize immunogenicity in humans.

Techniques for the production of single chain antibodies (U.S. Pat. No. 4,946,778) can also be adapted to produce single chain antibodies to polypeptides of this invention. Also, transgenic mice, or other organisms including other mammals, may be used to express humanized antibodies. See below.

Such antibodies can also be used for the immunoaffinity or affinity chromatography purification of the invention receptor proteins. Antibodies so produced can also be used, inter alia, in diagnostic methods and systems to detect the level of the invention receptor protein(s) present in a mammalian, preferably human, body sample, such as tissue. With respect to the detection of such receptor proteins, the antibodies can be used for in vitro diagnostic or in vivo imaging methods.

Numerous types of immunoassays are available and are known to those skilled in the art, e.g., competitive assays, sandwich assays, and the like, as generally described in, e.g., U.S. Pat. Nos. 4,642,285; 4,376,110; 4,016,043; 3,879,262; 3,852,157; 3,850,752; 3,839,153; 3,791,932; and Harlow and Lane, Antibodies, A Laboratory Manual, Cold Spring Harbor Publications, N.Y. (1988), each incorporated by reference herein.

Immunological procedures useful for in vitro detection of invention polypeptides in a sample include immunoassays that employ a detectable antibody. Such immunoassays include, for example, ELISA, Pandex microfluorimetric assay, agglutination assays, flow cytometry, serum diagnostic assays and immunohistochemical staining procedures, which are well known in the art. An antibody can be made detectable by various means well known in the art. For example, a detectable marker can be directly or indirectly attached to the antibody. Useful markers include, for example, radionucleotides, enzymes, fluorogens, chromogens and chemiluminescent labels.

In another aspect the invention concerns diagnostic methods and compositions. By means of having the human GPR54 receptor molecule and antibodies thereto, a variety of diagnostic assays are provided. For example, with antibodies, including monoclonal antibodies, to the invention receptor protein, the presence and/or concentration of the GPR54 in selected cells or tissues in an individual or culture of interest may be determined. These assays can be used in the diagnosis and/or treatment of various diseases attending a dysfunctional GPR54.

The above referenced anti-human GPR54 antibodies can also be used to modulate the activity of the invention receptor protein in living animals, in humans, or in biological tissues isolated therefrom. Accordingly, compositions comprising a carrier and an amount of an antibody having specificity for the invention receptor protein effective to block a native ligand or other ligands from binding to the invention receptor protein are contemplated herein. For example, a monoclonal antibody directed to an epitope of the invention receptor protein molecule and having an amino acid sequence substantially the same as an amino acid sequence as shown in SEQ ID NO: 2 may be useful for blocking binding of the invention receptor protein to a prospective ligand. The above applies equally to the mouse GPR54 receptor protein.

The above referenced antibodies receptors may also be employed to treat disease states attending the invention receptor proteins in a human.

Accordingly, methods are contemplated herein for detecting the presence of the novel receptor proteins on the surface of a cell. In one assay format human GPR54 receptor protein is identified and/or quantified by using labeled antibodies, preferably monoclonal antibodies which are reacted with body tissue known to express high levels hGPR54 and determining the specific binding thereto, the assay typically being performed under conditions conducive to immune complex formation. Unlabeled primary antibody can be used in combination with labels that are reactive with primary antibody to detect the receptor. For example, the primary antibody may be detected indirectly by a labeled secondary antibody made to specifically detect the primary antibody. Alternatively, the anti-GPR54 antibody can be directly labeled, as described above. A wide variety of labels may be employed, such as radionuclides, particles (e.g., gold, ferritin, magnetic particles, red blood cells), fluorophores, chemiluminescers, enzymes, enzyme substrates, enzyme cofactors, enzyme inhibitors, ligands (particularly haptens), etc.

Kits can also be supplied for use with the receptor of the subject invention in the detection of the presence of the receptor or antibodies thereto, as might be desired in the case of autoimmune disease. Thus, antibodies to GPR54, preferably monospecific antibodies such as monoclonal antibodies, or compositions of the receptor may be provided, usually in lyophilized form in a container, either segregated or in conjunction with additional reagents, such as anti-antibodies, labels, gene probes, polymerase chain reaction primers and polymerase, and the like.

Thus, in another aspect, the present invention relates to a screening kit for identifying agonists, antagonists, ligands, receptors, substrates, enzymes, etc. for the invention receptor protein; or compounds which decrease or enhance the production of such a receptor, which comprises:
(a) a receptor protein of the present invention;
(b) a recombinant cell expressing the invention receptor protein;
(c) a cell membrane expressing the invention receptor protein; or
(d) an antibody to the invention receptor protein; wherein the invention receptor protein is that of SEQ ID NO:2.

It will be appreciated that in any such kit, (a), (b), (c) or (d) may comprise a substantial component.

Screening Assays

As used herein, a cell which expresses GPR54 is one which contains that GPR as a functional receptor in its membrane; the cells may naturally express the GPR(s) of interest, or may be genetically engineered to express the GPR(s) of interest.

Human GPR54 proteins are hypothesized to be ubiquitous in the mammalian host and are responsible for many biological functions, including many pathologies.

Accordingly, it is desirous to find compounds and drugs which stimulate human GPR54 on the one hand and which can inhibit the function of human GPR54 on the other hand.

There are numerous methods for detecting ligand/receptor interaction. The most conventional are methods where the affinity of a receptor to a substance of interest is measured in radioligand binding assays. In these assays, one measures specific binding of a reference radiolabeled ligand molecule in the presence and in the absence of different concentrations of the compound of interest. The characteristic inhibition parameter of the specific binding of the reference radiolabeled ligand with the compound of interest, IC.sub.50, is taken as a measure of the affinity of the receptor to this compound (Weiland & Molinoff, 1981 and Swillens et all., 1995, are incorporated herein by reference). Recent advances in microchip sensor technology make it possible to measure direct interactions of a receptor molecule with a compound of interest in real time. This method allows for determination of both association and dissociation rate constants with subsequent calculation of the affinity parameter.

The proposed assays may simply test binding of a candidate compound wherein adherence to the cells bearing the receptor is detected by means of a label directly or indirectly associated with the candidate compound or in an assay involving competition with a labeled competitor. Further, these assays may test whether the candidate compound results in a signal generated by activation of the receptor, using detection systems appropriate to the cells bearing the receptor at their surfaces. Inhibitors of activation are generally assayed in the presence of a known agonist and the effect on activation by the agonist by the presence of the candidate compound is observed. Standard methods for conducting such screening assays are well understood in the art. Examples of potential Human GPR54 antagonists include antibodies or, in some cases, oligonucleotides or proteins which are closely related to the ligand of the Human GPR54, e.g., a fragment of the ligand, or small molecules which bind to the receptor but do not elicit a response, so that the activity of the receptor is prevented.

Still further the invention comprises novel assays for identifying functional ligands for hormone receptors.

The type of biological activity of the compounds, agonist or antagonist, may be determined in the cell based assays. In the methods described in Harpold & Brust, 1995, which is incorporated herein by reference, cells co-transfected with a receptor gene and a reporter gene construct, are used to provide means for identification of agonist and antagonist potential pharmaceutical compounds.

According to an aspect of the invention, DNA sequences are isolated that are suspected of encoding receptor proteins. These DNA sequences are transfected into a suitable receptor-deficient host cell that has been engineered to contain at least one reporter gene functionally linked to at least one operative hormone responsive element. The transfected receptor-deficient host cell (which now contains the suspected receptor and at least one reporter/HRE complex) is challenged with at least one candidate ligand(s) that can potentially bind with the ligand-binding domain region of the putative receptor protein encoded by the DNA sequence in question. The induction of the reporter gene is monitored by means of changes in the protein levels of the protein encoded by the reporter gene. Finally, a selection is made of ligand(s) that is capable of inducing production of the protein product of the reporter gene.

The present invention further provides a number of methods for utilizing the subject receptor proteins. One aspect of the present invention is a method for selecting new hormone analogues. The isolated receptor proteins of the invention by definition specifically bind ligands, although the exact nature and characteristics of the ligand is unknown at this time. Thus, the availability of the receptor proteins of the invention provide a means to screen for new molecules possessing the property of binding with high affinity to the ligand-binding region of the disclosed receptor proteins.

Thus, a binding domain of a structurally similar G protein-coupled receptor superfamily member may be used as a reagent to develop a binding assay. On one level, the binding domains can be used as affinity reagents for a batch or in a column selective process, to selectively retain ligands which bind. Alternatively, a functional assay is preferred for its greater sensitivity to ligand-binding. By using a reporter molecule for binding, either through a direct assay for binding, or through an expression or other functional linkage between binding and another function, an assay for binding may be developed. For example, by operable linkage of an easily assayable reporter gene to a controlling element responsive to binding by G protein-coupled receptor superfamily member, and where ligand-binding is functionally linked to protein induction, an extremely sensitive assay for the presence of a ligand or of a receptor results.

Accordingly, cells transformed with invention DNA (or RNA) can optionally be further transformed with a reporter gene expression construct, so as to provide a ready, indirect measure of the presence of functional human GPR54 receptor in the transformed cell. Such a reporter gene expression construct comprises:
a transcriptional control element; wherein the transcription control element, in the cell, is responsive to an intracellular condition that occurs when the human GPR54 receptor interacts with a compound having agonist or antagonist activity with respect to the receptor, and
a reporter gene encoding a transcription and/or translational product; wherein the product can be, directly or indirectly, readily measured; and wherein the gene is in operative association with the transcriptional control element.

Transcriptional control elements contemplated for use in this embodiment of the present invention include Ca²⁺ responsive enhancer elements such as the NF-AT enhancer.

Reporter genes contemplated for use in this embodiment of the present invention include the chloramphenicol transferase (CAT) gene, the gene product of which can be readily analyzed by a variety of methods known in the art. See, for example, Nielsen, et al., Anal. Biochem. 179, 19-23 (1989), luciferase and other enzyme detection systems such as alkaline phosphatase, .beta.-galactosidase, beta-lactamase and the like.

An aspect of the present invention contemplates the use of the disclosed receptor proteins in a screening process for identifying compounds which bind the human receptor and which would activate (agonists) or inhibit activation of (antagonists) the receptor protein of the present invention.

The receptor proteins of the invention may also be used to assess the binding of small molecule substrates and ligands in, for example, cells, cell-free preparations, chemical libraries, and natural product mixtures. These substrates and ligands may be natural substrates and ligands or may be structural or functional mimetics. See Coligan et al., Current Protocols in Immunology 1(2):Chapter 5 (1991).

The screening procedure can also be used to identify reagents such as antibodies which specifically bind to the receptor proteins of the invention and substantially affect its interaction with a ligand, for example.

In general, such screening procedures involve producing appropriate cells which express the receptor protein of the present invention on the surface thereof, followed by contacting the cells with a test compound to observe binding, or stimulation or inhibition of a functional response.

It will be readily appreciated by the skilled artisan that a receptor protein of the present invention may also be used in a method for the structure-based design of an agonist, antagonist or inhibitor of the receptor protein, by:
(a) determining in the first instance the three-dimensional structure of the receptor;
(b) deducing the three-dimensional structure for the likely reactive or binding site(s) of an agonist, antagonist or inhibitor;
(c) synthesizing candidate compounds that are predicted to bind to or react with the deduced binding or reactive site; and
(d) testing whether the candidate compounds are indeed agonists, antagonists or inhibitors.

It will be further appreciated that this will normally be an interactive process.

Accordingly, an embodiment of the invention provides screening assays conducted in vitro with cells which express the receptor proteins of the invention. The assay is based on the use of mammalian cell lines which express the receptor proteins (GPR54) functionally coupled to a mammalian G protein. In this assay, compounds are screened for their relative affinity as receptor agonists or antagonists by comparing the relative receptor occupancy to the extent of ligand induced stimulation or inhibition of second messenger metabolism. For example, activation of phospholipase C leads to increased inositol monophosphate metabolism. Means for measuring inositol monophosphate metabolism are generally described in Subers and Nathanson, J. Mol. Cell, Cardiol. 20:131-140 (1988), incorporated herein by reference.

Another screening technique includes the use of cells which express the invention receptor protein (for example, transfected CHO cells) in a system which measures extracellular pH or intracellular calcium changes caused by receptor activation. In this technique, compounds may be contacted with cells expressing the receptor protein of the present invention. A second messenger response, e.g., signal transduction, pH changes, or changes in calcium level, is then measured to determine whether the potential compound activates or inhibits the receptor.

Another method involves screening for receptor inhibitors by determining inhibition or stimulation of receptor-mediated cAMP and/or adenylate cyclase accumulation.

Such a method involves transfecting a eukaryotic cell with the invention receptor protein to express the receptor on the cell surface. The cell is then exposed to potential antagonists in the presence of the receptor of this invention. The amount of cAMP accumulation is then measured. If the potential antagonist binds the receptor, and thus inhibits receptor binding, the levels of receptor-mediated cAMP, or adenylate cyclase, activity will be reduced or increased.

Yet another method for detecting agonists or antagonists for the receptor proteins of the present invention is the yeast based technology as described in U.S. Pat. No. 5,482,835, the contents of which are incorporate by reference in their entirety herein.

Transgenic Non-Human Animals

The present invention further provides transgenic non-human mammals that are capable of expressing exogenous nucleic acids encoding the invention receptor proteins. As employed herein, the phrase “exogenous nucleic acid” refers to nucleic acid sequence which is not native to the host, or which is present in the host in other than its native environment (e.g., as part of a genetically engineered DNA construct). A transgenic mouse expressing exogenous invention nucleic acid encoding the invention receptor protein is particularly preferred.

Animal model systems which elucidate the physiological and behavioral roles of the invention receptor proteins are also contemplated, and may be produced by creating transgenic animals in which the expression of the invention receptor protein is altered using a variety of techniques. Examples of such techniques include the insertion of normal or mutant versions of nucleic acids encoding the invention polypeptide by microinjection, retroviral infection or other means well known to those skilled in the art, into appropriate fertilized embryos to produce a transgenic animal (Hogan et al., Manipulating the Mouse Embryo: A Laboratory Manual (Cold Spring Harbor Laboratory, (1986)).

Prophylactic and Therapeutic Methods

This invention also provides methods of treating an abnormal conditions related to both an excess of and insufficient amounts of Human GPR54 activity.

If the activity of Human GPR54 is in excess, several approaches are available. One approach comprises administering to a subject an inhibitor compound (antagonist) as hereinabove described along with a pharmaceutically acceptable carrier in an amount effective to inhibit activation by blocking binding of ligands to the Human GPR54, or by inhibiting a second signal and thereby alleviating the abnormal condition.

In another approach, expression of the gene encoding endogenous Human GPR54 receptor protein can be inhibited using expression blocking techniques.

Known such techniques involve the use of antisense sequences, either internally generated or separately administered. See, for example, O'Connor, J Neurochem (1991) 56:560 in Oligodeoxynucleotides as Antisense Inhibitors of Gene Expression, CRC Press, Boca Raton, Fla. (1988). Alternatively, oligonucleotides which form triple helices with the gene can be supplied. See, for example, Lee et al., Nucleic Acids Res (1979) 6:3073; Cooney et al., Science (1988) 241:456; Dervan et al., Science (1991) 251:1360. These oligomers can be administered per se or the relevant oligomers can be expressed in vivo.

For treating abnormal conditions related to an under-expression of Human GPR54 and its activity, several approaches are also available. One approach comprises administering to a subject a therapeutically effective amount of a compound which activates Human GPR54, i.e., an agonist as described above, in combination with a pharmaceutically acceptable carrier, to thereby alleviate the abnormal condition.

Alternatively, gene therapy may be employed to effect the endogenous production of Human GPR54 by the relevant cells in the subject. For example, a polynucleotide of the invention may be engineered for expression in a replication defective retroviral vector. Techniques for which are well known. For overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996).

Formulation and Administration

The soluble form of the invention receptor protein, and agonists and antagonist peptides or small molecules, may be formulated in combination with a suitable pharmaceutical carrier. Such formulations comprise a therapeutically effective amount of the receptor protein or compound, and a pharmaceutically acceptable carrier or excipient. Such carriers include but are not limited to, saline, buffered saline, dextrose, water, glycerol, ethanol, and combinations thereof. Formulation should suit the mode of administration, and is well within the skill of the art. The invention further relates to pharmaceutical packs and kits comprising one or more containers filled with one or more of the ingredients of the aforementioned compositions of the invention.

The receptor proteins and other compounds of the present invention may be employed alone or in conjunction with other compounds, such as therapeutic compounds.

Preferred forms of systemic administration of the pharmaceutical compositions include injection, typically by intravenous injection Other injection routes, such as subcutaneous, intramuscular, or intraperitoneal, can be used. Alternative means for systemic administration include transmucosal and transdermal administration using penetrants such as bile salts or fusidic acids or other detergents. In addition, if properly formulated in enteric or encapsulated formulations, oral administration may also be possible. Administration of these compounds may also be topical and/or localized, in the form of salves, pastes, gels and the like.

The dosage range required depends on the choice of peptide, the route of administration, the nature of the formulation, the nature of the subject's condition, and the judgment of the attending practitioner. Suitable dosages, however, are in the range of 0.1-100 .mu.g/kg of subject.

Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

The receptor proteins used in treatment can also be generated endogenously in the subject, in treatment modalities often referred to as “gene therapy” as described above. Thus, for example, cells from a subject may be engineered with a polynucleotide, such as a DNA or RNA, to encode a polypeptide ex vivo, and for example, by the use of a retroviral plasmid vector. The cells are then introduced into the subject.

Cells can be analyzed for the presence of the human GPR54 receptor lpha and beta subunit RNA in a variety of ways, such as for example, by Northern hybridization, slot blot analysis, and the like.

The examples below are carried out using standard techniques, which are well known and routine to those of skill in the art, except where otherwise described in detail. The examples illustrate, but do not limit the invention.

### EXAMPLE 1

I. Cloning of Human GPR54 cDNA Polynucleotide Sequences.

Initially, a search of the Human Genomic Database library (Genbank database htgs) was conducted using the entire coding region of the Rattus norvegicus receptor GPR54 as a template. The search yielded a piece of human genomic DNA (Genbank accession # AC023583) encoding human GPR54 receptor protein. Alignment of the above identified human genomic sequence with the above referenced rat GPR54 cDNA sequence, in turn, provided the putative start and stop codons of the corresponding human gPR54 encoding DNA.

Initially, the following specific oligonucleotide primers were utilized to generate a fragment for plasmid subcloning:

| | |
|---|---|
| HGPR54.F5 | 5′-AGC TGC CCT CTG GAC CCT GCG-3′ |
| HGPR54.R7 | 5′-CAA ACT TCA CAA CGA AAC TGC-3′ |

First-round PCR reaction was carried out using the primers pair HGPR54.F5 and HGPR54.R7 with the DNA polymerase Taq Gold (PE Biosystems, Poster City, Calif., USA) and the DNA template Marathon-ready human hypothalamus cDNA (Clontech, Inc., Palo Alto, Calif., USA) in the presence of 5% DMSO. The cycling parameters of the PCR were as follows:
95° C., 9 min, 1 cyc., 95° C., 20 sec., 55° C., 20 sec., 72° C., 1.5 min., 40 cyc.

The resulting PCR product was then used to as a template for a second-round PCR using the primer pair HGPR54.F6 and HGPR54.R8 under the same conditions except only 35 cycles were performed.

| | |
|---|---|
| HGPR54.F6 | 5′-CGA GCC CCT TCC TGA GTT CCA-3′ |
| HGPR54.R8 | 5′-CGA TTG GAT CCT CAC AAG AGA CCA AAA |
| | TAT TT-3′ |

The resulting (˜1,300 bp) PCR product from this nested PCR was purified and cloned into the vector pCR3.1 (Invitrogen, Carlsbad, Calif., USA) as described by the manufacturer and sequenced. Clones containing full-length GPR54 were thus identified. The complete coding sequence, the predicted polypeptide sequence, and the translation of human GPR54 receptor protein are shown in SEQ. ID. NOs. 1-3.

II. Cloning of Mouse GPR54 cDNA Polynucleotide Sequence.

Searching of the mouse genomic database using human GPR54 as a query sequence identified a piece of mouse genomic DNA (Genbank accession #AC073805) which appeared to contain mouse GPR54 Alignment of this mouse genomic sequence with the human and rat GPR54 cDNA sequences identified the putative start and stop codons of mouse GPR54.

To clone the complete coding sequence of mouse GPR54, four primers for PCR were designed and synthesized, as described below:

MGPR54.F1
5′-CTGGCAGGAAGAGAGCGACAG-3′
MGPR54.F2
5′-GGTAGCGGCCGCCACCATGGCCACCGAGGCGACATTGG-3′
MGPR54.R1
5′-CTTTACCCCACAGGCAGGACCG-3′
MGPR54.R2
5′-GCTTGGATCCTCAGAGTGAGGCAGTGCGTTC-3′

First-round PCR reaction was carried out using the primer pair MGPR54.F1 and MGPR54.R1 with the DNA polymerase Pfu Turbo (Stratagene, La Jolla, Calif., USA) and the DNA template Marathon-ready mouse hypothalamus cDNA (Clontech, Inc., Palo Alto, Calif., USA) in the presence of 5% DMSO. The cycling parameters of the PCR were as follows:
95° C., 2 min, 1 cyc., 95° C., 20 sec., 55° C., 20 sec., 72° C., 1.5 min., 35 cyc, 72° C., 7 min., 1 cyc.

The resulting PCR product was then used as template for a second-round PCR using the primer pair MGPR54.F2 and MGPR54.R2 under the same conditions except only 25 cycles were performed. The resulting ˜1,300 bp product from this nested PCR was purified and cloned into the vector pCR BluntII TOPO (Invitrogen, Carlsbad, Calif., USA) as described by the manufacturer and sequenced. Clones containing full-length mouse GPR54 were thus identified. The complete coding sequence, the predicted polypeptide sequence, and the translation of mouse GPR54 are shown in SEQ ID NOs; 4-6. The polypeptide sequences of human, mouse, and rat GPR54 are aligned as shown in FIG. 7.

### EXAMPLE 2

Identification of Agonists for Rat GPR54.

A. Activation of GPR54 by antho-RWamide I, NF1 and DF2 in the β-lactamase reporter enzyme assay.

The full-length coding sequence of rat GPR54 was sub-cloned into the expression vector pIRESpuromycin (Clontech, Palo Alto, Calif., USA) in accordance with the manufactures directions. This GPR54 plasmid was transfected into Aurora's CHO-NPAT-bla cells using the reagent Lipofectamine 2000 (GIBCO-BRL, Gaithersburg, Md., USA). Puromycin-resistant bulk stables expressing rat GPR54 were selected and used for screening for agonist.

β-lactamase assays were carried as described by (Zlokarnik et al., 1998, Quantitation of transcription and clonal selection of single living cells with beta-lactamase as reporter. Science 1998, 279:84-88). All peptides were from Phoenix Pharmaceuticals. Cells were seeded two days prior to assay. The day before being assayed, cells were changed to serum-free media. Four (4) hours after the addition of ligands, cells were loaded with dye and measured for fluorescence approximately 45 minutes later.

Three peptides, antho-RWamide I, neuropeptides NF1 and DF2 were found to activate rat GPR54 specifically in the primary screening. These peptides were then individually tested against GPR54-expressing cells. As shown in FIG. 8, the three neuropeptides activated rat GPR54 in a dose-dependent manner. Control cells (cells that do not express rat GPR54) showed no response to any of the three peptides (data not shown).

An extremely sensitive transcription-based assay is disclosed in Zlokarnik et al., 1998, Science 279:84-88 (Zlokarnik) and also in U.S. Pat. No. 5,741,657, both of which are incorporated by reference in their entirety herein. The assay disclosed in Zlokarnik and U.S. Pat. No. 5,741,657 employs a plasmid encoding β-lactamase under the control of an inducible promoter. This plasmid is transfected into cells together with a plasmid encoding a receptor for which it is desired to identify agonists. The inducible promoter on the β-lactamase is chosen so that it responds to at least one intracellular signal that is generated when an agonist binds to the receptor. Thus, following such binding of agonist to receptor, the level of β-lactamase in the transfected cells increases. This increase in β-lactamase is measured by treating the cells with a cell-permeable dye that is a substrate for cleavage by β-lactamase. The dye contains two fluorescent moieties. In the intact dye, the two fluorescent moieties are physically linked, and thus close enough to one another that fluorescence resonance energy transfer FRET) can take place between them. Following cleavage of the dye into two parts by β-lactamase, the two fluorescent moieties are located on different parts, and thus can diffuse apart. This increases the distance between the fluorescent moieties, thus decreasing the amount of FRET that can occur between them. It is this decrease in FRET that is measured in the assay.

A variety of 3-lactamases are known in the art and are suitable for use in the present methods. One particularly well-studied form of β-lactamase is the product of the Amp^{r} gene of E. coli, TEM-1 β-lactamase (Sutcliffe, 1978, Proc. Natl. Acad. Sci. USA 75:3737-3741). A version of TEM-1, with its signal sequence deleted so that it accumulates in the cytoplasm, is disclosed in Kadonaga et al., 1984, J. Biol. Chem. 259:2149-2154. β-lactamases are produced by a variety of bacteria and many β-lactamases have been well studied. For example, Staphlyococcus aureus produces PC1 β-lactamase; Bacillus cereus produces a β-lactamase known as β-lactamase I; Escherichia coli produces RTEM 3-lactamase (Christensen et al., 1990, Biochem J. 266:853-861. All that is necessary for a particular 3-lactamase to be suitable for use in the present invention is that it be capable of cleaving the fluorescent substrate in such a way that the two fluorescent moieties of the substrate can diffuse away from each other following cleavage. This can be easily tested and thus the suitability of a particular β-lactamase can be easily determined.

The amino acid sequences of a variety of suitable β-lactamases are disclosed in Ambler, 1980, Phil. Trans. R. Soc. Lond. (Ser. B.) 289:321-331. One of skill in the art can readily synthesize synthetic DNA sequences that encode these β-lactamases. Alternatively, these β-lactamases can be cloned from natural sources. DNA sequences encoding β-lactamases can be placed into suitable expression vectors and transfected into cells for use in the methods of the present invention. A DNA sequence encoding a particular β-lactamase that can be used in the methods of the present invention is shown in SEQ.ID.NO.:1 of U.S. Pat. No. 5,741,657 while the corresponding amino acid sequence is shown as SEQ.ID.NO.:2 of U.S. Pat. No. 5,741,657. A plasmid containing this DNA (pTG2del1) is described in Kadonaga et al., 1984, J. Biol. Chem. 259:2149-2154.

Moore et al., 1997, Anal. Biochem. 247:203-209 describes a method for engineering a form of RTEM1-lactamase that is maintained intracellularly by eukaryotic cells. DNA encoding the native signal sequence of RTEM1 β-lactamase is removed and replaced with a methionine codon. Sequences that provide for optimal translational efficiency in eukaryotes are placed immediately upstream of this methionine by PCR. This modified 13-lactamase coding sequence is then cloned into expression vector pRc-CMV (Invitrogen, San Diego, Calif.). This places the coding sequences under the control of the human intermediate early cytomegalovirus promoter and provides a bovine growth hormone polyadenylation sequence. This construct, known as pCMV-BL, was able to direct the expression of active β-lactamase in the cytoplasm of mammalian cells.

A preferred embodiment of the present invention makes use of the fluorescent β-lactamase substrate used in the assays for transcriptional activation described by Zlokarnik et al., 1998, Science 279:84-88.

(B) Functional activation of human GPR54 receptor protein by the neuropeptides NF1, DF2 and antho-RWamide I in the aequorin assay.

The HEK293/aeq17 cell line was licensed in from NIH (Button and Brownstein, 1993, Cell Calcium, 14:663-671). The cells were grown in Dulbecco's Modified Medium (MEM, GIBCO-BRL, Gaithersburg, Md., USA)+10% fetal bovine serum (heat inactivated), 1 mM sodium pyruvate, 500 ug/ml Geneticin, 100 ug/ml streptomycin, 100 units/ml penicillin. Human GPR54 was cloned into the vector pIRESpuromycin (Clontech, Inc., Palo Alto, Calif., USA) and transfected into HBEK293/aeq17 using Lipofectamine-2000 (Gaithersburg, Md., USA) following the conditions suggested by GIBCO-BRL. Puromycin-resistant clones were selected and bulk stables were used for further analysis.

For the aequorin assay, cell were washed once with DMEM+0.1% fetal bovine serum, and then charged for one hour at 37° C. 15% CO₂ in DMEM containing 8 uM coelenterazine cp (Molecular Probes, Eugene, Oreg., USA) and 30 uM glutathione. The cells were then washed once with Versene (GIBCO-BRL, Gaithersburg, Md., USA), detached using Enzyme-free cellissociation buffer (GIBCO-BRL, Gaithersburg, Md., USA), diluted into ECB (Ham's P12 nutrient mixture (GIBCO-BRL)+0.3 mM CaCl2, 25 mM HEPES, pH7.3, 0.1% fetal bovine serum). The cell suspension was centrifuged at 500×g for 5 min. The supernatant was removed, and the pellet was then resuspended in 10 mL ECB. The cell density was determined by counting with a hemacytometer and adjusted to 500,000 cells/ml in ECB.

The neuropeptides were diluted in ECB into 2× concentrates using 5-fold serial dilutions, and aliquoted into assay plates in triplicates at 0.1 ml/well. The cell suspension was injected at 0.1 ml/well, read and integrated for a total of 400 readings using a luminometer (Luminoskan Ascent, Labsystems Oy, Helsinki, Finland). Data was analyzed using the software GraphPad Prism Version 3.0 (GraphPad Software, Inc., San Diego, Calif., USA). As shown in FIG. 9, cells expressing Human GPR54 receptor showed robust, dose-dependent response to antho-RWamide I, antho-RW amide II, and two peptides modified from NF1.

### EXAMPLE 3

Mammalian Cell Expression

The receptors of the present invention can also be expressed in either human embryonic kidney 293 (HEK293) cells or adherent dhfr CHO cells. To maximize receptor expression, typically all 5′ and 3′ untranslated regions (UTRs) are removed from the receptor cDNA prior to insertion into a pCDN or pCDNA3 vector. The cells can there after be transfected with individual receptor cDNAs by lipofectin and selected in the presence of appropriate amounts of (ca 400 mg/ml) G418.

After a suitable period of time, i.e., about 3 weeks of selection, individual clones are picked and expanded for further analysis. HEK293 or CHO cells transfected with the vector alone serve as negative controls. To isolate cell lines stably expressing the individual receptors, about 24-36 clones are typically selected and analyzed by Northern blot analysis. Receptor mRNAs are generally detectable in about 50% of the G418 resistant clones analyzed.

### EXAMPLE 4

Ligand Bank for Binding and Functional Assays

A bank of over 200 putative receptor ligands may be assembled for screening. The bank MAY comprise: transmitters, hormones and chemokines known to act via a human G protein-coupled receptor; naturally occurring compounds which may be putative agonists for a human G protein-coupled receptor; non-mammalian, biologically active peptides for which a mammalian counterpart has not yet been identified; and compounds not found in nature, but which activate G protein-coupled receptors with unknown natural ligands. This bank is used to initially screen the receptor for known ligands, using both functional (i.e. calcium, cAMP, microphysiometer, oocyte electrophysiology, etc, see below) as well as binding assays.

### EXAMPLE 5

Ligand Binding Assays

Ligand binding assays provide a direct method for ascertaining receptor pharmacology and are adaptable to a high throughput format. The purified ligand for a receptor is radiolabeled to high specific activity (50-2000 Ci/mmol) for binding studies. A determination is then made that the process of radiolabeling does not diminish the activity of the ligand towards its receptor. Assay conditions for buffers, ions, pH and other modulators such as nucleotides are optimized to establish a workable signal to noise ratio for both membrane and whole cell receptor sources. Such conditions are well known to one skilled in the art.

For these assays, specific receptor binding is defined as total associated radioactivity minus the radioactivity measured in the presence of an excess of unlabeled competing ligand. Where possible, more than one competing ligand is used to define residual nonspecific binding.

### EXAMPLE 6

Functional Assay in Xenopus Oocytes

Capped RNA transcripts from linearized plasmid templates encoding the receptor cDNAs of the invention may be synthesized in vitro with RNA polymerases in accordance with standard procedures. In vitro transcripts are suspended in water at a final concentration of 0.2 mg/ml. Ovarian lobes can be removed from adult female toads, stage V defolliculated oocytes are obtained, and RNA transcripts (10 ng/oocyte) are injected in a 50 nl bolus using a microinjection apparatus.

Thereafter, two electrode voltage clamps are used to measure the currents from individual Xenopus oocytes in response to agonist exposure. Recordings are made in Ca²⁺ free Barth's medium at room temperature. The Xenopus system can be used to screen known ligands and tissue/cell extracts for activating ligands.

### EXAMPLE 7

Microphysiometric Assays

Activation of a wide variety of secondary messenger systems results in extrusion of small amounts of acid from a cell. The acid formed is largely as a result of the increased metabolic activity required to fuel the intracellular signaling process. The pH changes in the media surrounding the cell are very small but are detectable by the CYTOSENSOR microphysiometer (Molecular Devices Ltd., Menlo Park, Calif.). The CYTOSENSOR is thus capable of detecting the activation of a receptor which is coupled to an energy utilizing intracellular signaling pathway such as the G-protein coupled receptor of the present invention.

### EXAMPLE 8

Extract/Cell Supernatant Screening

A large number of mammalian receptors exist for which there remains, as yet, no cognate activating ligand (agonist). Thus, active ligands for these receptors may not be included within the ligands banks as identified to date.

Accordingly, the Human and/or mouse GPR54 receptor(s) of the invention may also be functionally screened (using calcium, cAMP, microphysiometer, oocyte electrophysiology, etc., functional screens) against tissue extracts to identify natural ligands. Extracts that produce positive functional responses can be sequentially subfractionated until an activating ligand is isolated and identified.

### EXAMPLE 9

Calcium and cAMP Functional Assays

G protein-coupled receptors which are expressed in HEK 293 cells have been shown to be coupled functionally to activation of PLC and calcium mobilization and/or cAMP stimulation or inhibition. Basal calcium levels in the HEK 293 cells in receptor-transfected or vector control cells ere observed to be in the normal, 100 nM to 200 nM, range. HEK 293 cells expressing recombinant receptors may then be loaded with fura 2 and in a single day>150 selected ligands or tissue/cell extracts can be evaluated for agonist induced calcium mobilization.

Similarly, HEK 293 cells expressing recombinant receptors are evaluated for the stimulation or inhibition of cAMP production using standard cAMP quantitation assays. Agonists presenting a calcium transient or cAMP fluctuation are tested in vector control cells to determine if the response is unique to the transfected cells expressing receptor.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims.

Summary of Sequences

SEQ. ID. NO: 1 is the nucleotide sequence encoding a human GPR54 receptor protein.

SEQ. ID. NO: 2 is the deduced amino acid sequence of the human GPR54 receptor protein.

SEQ. ID. NO: 3 is the translation sequence of the open reading frame of the gene encoding the human GPR54 receptor protein.

SEQ. ID. NO: 4 is the nucleotide sequence encoding a mouse GPR54 receptor protein.

SEQ. ID. NO: 5 is the deduced amino acid sequence of the mouse GPR54 receptor protein.

SEQ. ID. NO: 6 is the translation sequence of the open reading frame of the gene encoding the mouse GPR54 receptor protein.

1. An isolated nucleic acid molecule, comprising a sequence of nucleotides that encodes a human G protein-coupled receptor—GPR54, wherein the sequence of nucleotides is selected from the group consisting of:
(a) a sequence of nucleotides that encodes a human GPR54 receptor protein and comprises the sequence of nucleotides set forth in SEQ ID NO:1;
(b) a sequence of nucleotides that encodes human GPR54 receptor protein and that hybridizes under conditions of high stringency to the complement of the sequence of nucleotides set forth in SEQ ID NO: 1; and, if it is DNA, is fully complementary or, if it is RNA, is identical to mRNA native to a human cell;
(c) a sequence of nucleotides degenerate with the human GPR54 receptor protein encoding sequence of (a) or (b).

2. An isolated nucleic acid molecule, comprising a coding region that encodes a splice variant of a human GPR54 receptor, wherein the human GPR54 receptor protein is encoded by a sequence of nucleotides as set forth in SEQ. ID No. 1.

3. The isolated nucleic acid molecule according to claim 1, wherein the isolated nucleic acid molecule is genomic DNA.

4. The isolated nucleic acid molecule according to claim 1, wherein the isolated nucleic acid molecule is mRNA.

5. The isolated nucleic acid molecule according to claim 1, wherein the isolated nucleic acid molecule is cDNA.

6. An isolated nucleic acid molecule that encodes a human GPR54 receptor protein having an amino acid sequence as set forth in SEQ ID NO:2.

7. An isolated polypeptide encoded by a nucleotide sequence that is a splice variant of a isolated nucleic acid molecule that encodes a protein comprising the amino acid sequence set forth in SEQ ID NO:2.

8. Isolated cells, comprising the nucleic acid molecule of claim 1, wherein the cells are bacterial cells, mammalian cells or amphibian oocytes, and the nucleic acid molecule is heterologous to the cells.

9. An isolated human GPR54 receptor protein encoded by the nucleic acid molecule of claim 1.

10. A method for identifying a functional human GPR54 receptor protein in a biological sample, the method comprising:
(a) introducing the nucleic acid molecule of claim 1 into a suitable host cell that expresses a functional GPR54 receptor protein receptor; and
(b) assaying for second messenger activity in cells of step (a).

11. A method for identifying DNA sequences encoding a human GPR54 receptor protein, the method comprising probing a cDNA library or a genomic library with a labeled probe comprising the nucleotide sequence of SEQ ID NO: 1, and recovering from the library those sequences having a significant degree of homology relative to the probe.

12. A method for identifying a human GPR54 receptor protein, comprising:
(a) introducing the nucleic acid molecule of claim 1 into eukaryotic cells; and
(b) detecting second messenger activity in the cells of step (a), wherein the activity is mediated by a polypeptide encoded by the introduced nucleic acid molecule.

13. A method for detecting human GPR54 receptor protein messenger RNA in a biological sample comprising the steps of:
(a) contacting all or part of the nucleic acid sequence shown in SEQ ID NO:1 with the biological sample under conditions allowing a complex to form between the nucleic acid sequence and the messenger RNA
(b) detecting the complexes; and
(c) determining the level of the messenger RNA.

14. A bioassay for identifying a test compound, which modulates the activity of a human GPR54 receptor protein, the bioassay comprising:
(a) measuring the second messenger activity of eukaryotic cells transformed with DNA encoding the human GPR54 receptor protein in the absence of the test compound, thereby obtaining a first measurement;
(b) measuring the second messenger activity of eukaryotic cells transformed with DNA encoding the human GPR54 receptor protein in the presence of the test compound, thereby obtaining a second measurement; and
(c) comparing the first and second measurement and identifying those compounds that result in a difference between the first measurement and the second measurement as a test compound that modulates the activity of the human GPR54 receptor protein, wherein the eukaryotic cells express a functional human parathyroid hormone-2 receptor protein.

15. A method for following progress of a therapeutic regime designed to alleviate a condition characterized by abnormal expression of a gene product of the isolated nucleic acid molecule of claim 1, comprising:
(a) assaying a sample from a subject to determine level of a parameter selected from the group consisting of (i) a polypeptide encoded by a the nucleotide sequence of SEQ ID NO: 1 and (ii) a polypeptide having the amino acid sequence as set forth in SEQ ID NO: 3, at a first time point;
(b) assaying level of the parameter selected in (a) at a second time point and
(c) comparing the level at the second time point to the level determined in (a) as a determination of effect of the therapeutic regime.

16. A method for determining regression, progression or onset of a pathological disorder characterized by a dysfunctional signal transduction comprising incubating a sample obtained from a patient with the disorder with a complimentary nucleic acid hybridization probe having a sequence of nucleotides that are substantially homologous to those of SEQ ID NO: 1 and determining binding between the probe and any complimentary mRNA that may be present in the sample as determinative of the regression, progression or onset of the pathological disorder in the patient.

17. The method of claim 14, wherein the sample is a biological tissue.

18. A method for determining regression, progression or onset of a pathological disorder characterized by a dysfunctional signal transduction comprising: contacting a sample, from a patient with the disorder, with a detectable probe that is specific for the gene product of the isolated nucleic acid molecule of claim 1, under conditions favoring formation of a probe/gene product complex, the presence of which is indicative of the regression. progression or onset of the pathological disorder in the patient.

19. The method of claim 18, wherein the probe is an antibody.

20. The method of claim 19, wherein the antibody is labeled with a radioactive label or an enzyme.

21. A pharmaceutical composition comprising the polypeptide according to claim 6 in combination with a pharmaceutically acceptable carrier, diluent or excipient.

22. A method for preventing or delaying onset of a condition associated with reduced or non-existent levels of the polypeptide of claim 9 in a subject prone thereto comprising administering an effective amount of the polypeptide to the subject sufficient to prevent or delay onset of the condition.

23. A method for monitoring the efficacy of an agent in correcting an abnormal level of the polypeptide of claim 9 in a subject prone thereto, comprising administering an effective amount of the agent to the subject and determining a level of the polypeptide in the subject following its administration, wherein a change in the level of the polypeptide towards a normal level is indicative of the efficacy of the agent.

24. A method for detecting a binding partner for the a Human GPR54 receptor protein in a sample suspected of containing the binding partner, comprising:
(i) contacting the sample with the GPR54 receptor under conditions favoring binding of the receptor to the binding partner;
(ii) determining presence of the binding partner in the sample by detecting binding of the receptor to the binding partner.

25. A method of modulating the endogenous signal transducing activity of a GPR54 receptor protein in a mammal comprising administering to the mammal an effective amount of the binding partner identified in claim 24.

26. The isolated polynucleotide of claim 1 comprising a nucleotide sequence encoding a polypeptide which has at least 80% identity to the amino acid sequence of SEQ ID NO:2, which may include up to Na amino acid alterations over the entire length of SEQ ID NO: 2, wherein Na is the maximum number of amino acid alterations, and is calculated by the formula
Nₐ=Xₐ−(XₐY),
in which Xₐ is the total number of amino acids in SEQ ID NO:2, and Y has a value of 0.80, wherein any non-integer product of Xₐ and Y is rounded down to the nearest integer prior to subtracting such product from Xₐ.

27. A method for identifying a ligand(s) that activates an receptor protein, the method comprising:
(a) introducing a G protein-coupled receptor protein into receptor protein-deficient cells, wherein the cells contain a reporter gene functionally linked to a hormone response element responsive to the reporter gene;
(b) challenging the cells with candidate ligand(s) which can potentially bind with the ligand-binding domain of the receptor protein;
(c) monitoring induction of the reporter gene (s), thereby identifying ligand(s) that activate the receptor protein.

28. A method for identifying ligand(s) that activate an receptor protein, the method comprising:
contacting endogenous-receptor protein-deficient host cells with candidate ligand(s) wherein the host cells contain a reporter gene functionally linked to a hormone response element, and an exogenous gene encoding an receptor protein, wherein the hormone response element, upon activation, induces expression of the reporter gene(s);
monitoring induction of the reporter gene(s); and
identifying ligand(s) that activate the receptor protein.

29. Method for screening for a disorder characterized by expression of a dysfunctional human GPR54 receptor protein coded for by a cDNA comprising a sequence of nucleotides substantially homologous to those set forth in SEQ. ID. NO: 1, the method comprising the steps of contacting a sample from a subject believed to suffer from the disorder with an antibody specific for an expression product of SEQ ID NO:1, and determining binding between the antibody and the expression production as an indication of possible presence of the disorder in the subject.

30. Method for screening for a disorder characterized by expression of a dysfunctional human GPR54 receptor protein encoded by a cDNA molecule comprising a sequence of nucleotides substantially homologous to those set forth in SEQ. ID. NO: 1, comprising contacting a cDNA or mRNA containing sample from a subject with a nucleic acid hybridization probe which hybridizes to a cDNA molecule comprising a sequence of nucleotides as set forth in SEQ ID NO: 1, and determining binding of the hybridization probe to the cDNA or mRNA as an indication of possible presence of the disorder in the subject.

31. An antibody that is specific for the polypeptide of claim 9.

32. The antibody according to claim 31, wherein the antibody is a monoclonal antibody.

33. A method for identifying agonist or antagonist of a Human or mouse GPR54 receptor protein comprises:
contacting a cell expressing on the surface thereof the receptor protein, wherein the receptor is associated with a second component capable of providing a detectable signal in response to the binding of a compound to the receptor, with a compound to be screened under conditions favoring binding of the compound top the receptor protein; and
determining whether the compound binds to and activates or inhibits the receptor protein by measuring the level of a signal generated from the interaction of the compound with the receptor protein.

34. A suitable host cell transfected with an isolated nucleic acid molecule comprising a sequence of nucleotides or ribonucleotides that encodes a Human or Mouse GPR54 receptor protein.

35. A recombinant non-human cell line which has been engineered to express a heterologous protein, the cell line comprising a host cell transformed or transfected with a heterologous nucleic acid molecule comprising a sequence of nucleotides or ribonucleotides that inducibly express a Human or Mouse GPR54 receptor protein.

36. An isolated cell transformed or transfected with a sequence of nucleotides or ribonucleotides under conditions favoring cell surface expression of a functional Human or Mouse GPR54 receptor protein.

37. An expression vector comprising the nucleic acid molecule of claim 1, operably linked to a regulatory nucleotide sequence that controls expression of the nucleic acid molecule in a host cell.

38. An isolated nucleic acid molecule, comprising a sequence of nucleotides that encodes a mouse G protein-coupled receptor—GPR54, wherein the sequence of nucleotides is selected from the group consisting of:
(a) a sequence of nucleotides that encodes a mouse GPR54 receptor protein and comprises the sequence of nucleotides set forth in SEQ ID NO:4;
(b) a sequence of nucleotides that encodes mouse GPR54 receptor protein and that hybridizes under conditions of high stringency to the complement of the sequence of nucleotides set forth in SEQ ID NO:4; and, if it is DNA, is fully complementary or, if it is RNA, is identical to mRNA native to a human cell;
(c) a sequence of nucleotides degenerate with the mouse GPR54 receptor protein encoding sequence of (a) or (b).

39. An isolated nucleic acid molecule, comprising a coding region that encodes a splice variant of a mouse GPR54 receptor, wherein the mouse GPR54 receptor protein is encoded by a sequence of nucleotides as set forth in SEQ ID NO: 4.

40. The isolated nucleic acid molecule according to claim 38, wherein the isolated nucleic acid molecule is genomic DNA.

41. The isolated nucleic acid molecule according to claim 38, wherein the isolated nucleic acid molecule is mRNA.

42. The isolated nucleic acid molecule according to claim 38, wherein the isolated nucleic acid molecule is cDNA.

43. An isolated nucleic acid molecule that encodes a mouse GPR54 receptor protein having an amino acid sequence as set forth in SEQ ID NO:5.

44. An isolated polypeptide encoded by a nucleotide sequence that is a splice variant of a isolated nucleic acid molecule that encodes a protein comprising the amino acid sequence set forth in SEQ ID NO:5.

45. Isolated cells, comprising the nucleic acid molecule of claim 38, wherein the cells are bacterial cells, mammalian cells or amphibian oocytes, and the nucleic acid molecule is heterologous to the cells.

46. An isolated mouse GPR54 receptor protein encoded by the nucleic acid molecule of claim 1.
